(19) United States
(12) Patent Application Publication
(10) Pub. No.: US 2011/0281352 A1
(43) Pub. Date: Nov. 17, 2011

(54) Transplant Storage

(75) Inventors: Sten Raeder, Oslo (NO); Tor Paaske Utheim, Oslo (NO)

(73) Assignee: MEDINNOVA AS, Oslo (NO)

(21) Appl. No.: 12/597,546

(22) PCT Filed: Apr. 28, 2008
(86) PCT No.: PCT/EP2008/003733

§ 371 Date: Sep. 3, 2010

Related U.S. Application Data
(60) Provisional application No. 60/914,127, filed on Apr. 26, 2007.
(60) Provisional application No. 60/989,655, filed on Nov. 21, 2007.

(51) Int. Cl. C12N 5/071; C12M 3/00; C12N 5/077

(52) U.S. Cl. 435/374; 435/307.1

(57) ABSTRACT

A kit (1) for storing cells or tissues. The kit (1) comprises a frame (7) having an opening therein and a peripheral wall surrounding the opening. Also provided is a sealable receptacle (2) for receiving the frame (7) and for receiving a liquid medium (6). A section of the sealable receptacle (2) being formed of a resilient member (5) for permitting access to the interior of the receptacle (5) by a penetrating element and subsequently forming a seal after withdrawal of the penetrating element.

### FIELD OF THE INVENTION

The present invention relates to a kit for storing cells or tissues and the use of the kit for storing cells or tissues such as for transplantation or implantation. The present invention also relates to a method of storing cells or tissues including limbal epithelial cells, conjunctival cells, corneal endothelial cells, retinal cells, mucosal cells, epidermal cells (i.e. skin), or bone marrow derived cells. The present invention further relates to a method of providing limbal cell explants and to a cell storage medium.

### BACKGROUND

The cornea serves as the window of the eye, and consists of three layers, of which the outer most is called epithelium. In the very periphery of the cornea, that is the transition zone between the translucent and the white part of the eye, also called the limbus, the stem cells of the cornea are located. These cells may suffer from different types of diseases and insults affecting the cornea, giving rise to a condition called limbal stem cell deficiency. This condition can lead to blindness as the cornea becomes opaque.

Transplantation of ex vivo expanded human limbal epithelial cells (HLEC) is a therapy for limbal stem cell deficiency (LSCD).²⁰⁻²⁵ The principle of ex vivo expansion of HLEC is to generate an undifferentiated corneal epithelium from a biopsy of functional limbal tissue harvested from the patient (autograft) or a living related donor or a cadaver eye (allograft). HLEC may be cultured ex vivo by a variety of expansion protocols including limbal explant culture,²⁶⁻²⁹ cell suspension culture,^{20, 26, 30, 31} culture on intact^{28, 29, 32, 33} or epithelially denuded^{26, 31-35} amniotic membranes (AM) or other cell culture surfaces, ^{20, 26, 36-40} cocultivation with lethally irradiated 3T3 fibroblasts,^{20, 22, 27, 30} and air-lifting. ^{23, 41.} An alternative approach in treating LSCD, has been the use of autologous oral mucosal epithelial sheets.⁴²⁻⁴⁵ Although the protocols have shown good clinical outcomes, limbal epithelial stem cell therapy still faces challenges regarding surgery logistics, tissue sterility, tissue transportation, and availability of tissue. The timing of surgery may be complicated as the engineering of multilayered epithelia requires culture periods of 3-4 weeks, and the tissue cultures are susceptible to microbial contamination during the setup of the cultures, medium change, and transportation to the operating theatre. The clinical application of limbal epithelial stem cell therapy is currently limited to ophthalmology departments with the knowledge and laboratory facilities available for tissue engineering.

There are several problems that arise in the storage of limbal epithelial cells at present.

Firstly, limbal epithelial cells are typically stored at present by culturing on an amniotic membrane which has been sutured onto a polyester membrane carrier. The membrane carrier, together with the amniotic membrane and cultured epithelial cells are then immersed in organ culture medium (generally stored in a stoppered bottle.). The problem with this approach is that it is time consuming and fiddly to fasten the amniotic membrane to the membrane carrier. Furthermore, once the membrane carrier is immersed in the medium, it cannot readily be subjected to microbiological assessment without opening the stoppered bottle which risks contamination of the medium.

A second problem is in the mechanical nature of the storage of the limbal epithelial cells. Existing approaches for storing the epithelial cells result in the amniotic membrane to which the cells were applied floating at liberty within the culture medium. However, in practice, this provides no protection for the epithelial cells if they are transported. The transport of cultured limbal epithelial cells is important because, in practice, it is efficient for cells to be stored at “eye banks” and then transported to a hospital where the implantation procedure is carried out. Furthermore, the depth of medium in which the cultured limbal epithelial cells are immersed may affect the storage and development of the cells. Therefore, allowing the amniotic membrane, to which the cells have been applied, to float freely within the medium may subject the cells to sub-optimal conditions.

A third problem in the storage of cultured limbal epithelial cells is the temperature at which they are stored. Residual corneoscleral donor rims following penetrating keratoplasty, which are a source of HLEC for the engineering of cultured corneal epithelium,⁴⁷⁻⁵⁰ are generally stored in OC media⁵¹ at temperatures between 31° C. and 37° C. (European Eye Bank Association Directory, 2007), or in Optisol-GS⁵² (Bausch & Lomb, Irvine, Calif.) at 4° C. Furthermore, storage of limbal epithelium in Optisol-GS has been shown to produce a basal layer cell viability of 95% after six days.⁵³ However, special equipment is required to store explants at such temperatures.

As mentioned above, HLEC are generated by obtaining a biopsy of functional limbal tissue harvested from a donor. However, the circumferential location of the biopsy site is poorly reported in the prior art. Therefore it is has not previously been known whether biopsies from certain locations have improved qualities.

The present invention seeks to alleviate one or more of the above problems.

Example 1 herein reports for the first time a method for short-term eye bank storage of cultured HLEC, which may be beneficial in limbal epithelial stem cell therapy.⁴⁶ In the study, 3-weeks HLEC cultures were transferred from the incubator to a glass container with organ culture (OC) medium and stored for one week at 23° C., while maintaining the original multilayered structure and undifferentiated phenotype (FIG. 8). The experimental design of this method has several advantages. Firstly, the maintenance of the limbal phenotype offers flexibility in scheduling the transplantation. Secondly, tissue storage allows time to perform microbiological testing of the storage media, which may enhance the safety of transplantation of ex vivo expanded HLEC. Thirdly, the closed system enables tissue to be transported from the laboratory to the operating theatre and between eye banks to increase the availability of tissue. Finally, storage at room temperature eliminates the need for heating cabinets.

### SUMMARY OF THE INVENTION

According to one aspect of the present invention there is provided a kit for storing cells or tissue comprising:

a frame having an opening therein and a peripheral wall surrounding the opening; and

a sealable receptacle for receiving the frame and for receiving a liquid medium, a section of the sealable receptacle being formed of a resilient member for permitting access to the interior of the receptacle by a penetrating element and subsequently forming a seal after withdrawal of the penetrating element.

Conveniently, the kit is suitable for storing a substrate for culturing cells or tissue.

Preferably, the substrate is a planar or arcuate substrate.

Conveniently, the substrate is an amniotic membrane, a contact lens, a collagen gel or a plastics material, preferably wherein the amniotic membrane is located on a supporting mesh.

Preferably, the cells are: limbal cells, conjunctival cells, endothelial cells, mucosal cells, retinal cells, bone marrow derived cells or epidermal cells.

Conveniently, the limbal cells, conjunctival cells, corneal endothelial cells, retinal cells, mucosal cells, epidermal cells on bone marrow derived cells are cultured cells.

Advantageously, the kit further comprises an elongate or annular resilient element locatable about the peripheral wall for securing a planar substrate across the opening in the frame.

Conveniently, the peripheral wall contains a circumferential furrow for receiving the elongate or annular resilient element.

Preferably, the kit further comprises at least one float attachable to the frame for supporting the frame in the medium.

Advantageously, the float is attachable to the exterior of the peripheral wall.

Conveniently, the frame comprises a circumferential groove for receiving the float

Preferably, the float or floats are located around the peripheral wall such that when the frame is located within the receptacle and the float or floats are supporting the frame in the medium, float or floats are interposed between the peripheral wall and the receptacle.

Conveniently, the greatest distance between any part of the float or floats forms a maximum diameter and the maximum diameter is at least 80%, preferably at least 90% of the minimum diameter of the receptacle.

Preferably, the at least one float is made from an impact absorbing material.

Advantageously, the at least one float is made from a deformable material which is capable of being punctured by the penetrating element.

Conveniently, a gap is provided in the float or floats for receiving the penetrating element.

Preferably, the float is locatable on the frame such that a planar substrate supported on the frame will lie less than 2 mm below the level of the liquid medium, preferably less than 1 mm.

Advantageously, the kit further comprises a support mechanism for holding the receptacle and permitting free rotation of the receptacle relative to at least a portion of the support mechanism.

Conveniently, the support mechanism comprises a gimbal. The gimbal may allow rotation in one, two or three perpendicular axes.

Alternatively, the support mechanism comprises a spherical inner casing for holding the receptacle and an outer casing comprising a spherical recess for receiving the inner casing, the inner casing being rotatable within the outer casing.

Preferably, the receptacle comprises a removable cap.

Advantageously, the removable cap is attached to the receptacle by a hinge.

Conveniently, the resilient member is located in the cap.

Preferably, the frame is a hollow cylinder.

Advantageously, the peripheral wall comprises one or more apertures for allowing passage of the medium therethrough.

Conveniently, the kit further comprises the medium.

Preferably, the medium is organ culture medium.

Alternatively, the medium is serum free medium CnT-20.

Advantageously, the medium comprises minimal essential medium.

Conveniently, the medium is a serum based medium.

Preferably, the medium comprises fetal bovine serum.

Alternatively, the medium is a serum-free medium.

Conveniently, the serum-free medium comprises: Optisol-GS or PAA-Quantum.

Alternatively, the serum-free medium comprises: a buffering agent and minimal essential medium.

Preferably the buffering agent comprises HEPES (4-(2-hydroxyethyl)-1-piperazineethane-sulfonic acid), more preferably at 25 mM concentration.

Advantageously, the minimal essential medium comprises amino acids, salts, glucose and vitamins.

Conveniently, the salts comprise at least one of potassium chloride, magnesium sulphate, sodium chloride and sodium dihydrogen phosphate and/or the vitamins comprise at least one of folic acid, nicotinamide, riboflavin and B-12.

Advantageously, the medium comprises sodium bicarbonate.

Conveniently, the medium comprises an antibiotic.

Preferably, the antibiotic is gentamicin, vancomycin, amphotericin B or mixtures thereof.

Advantageously, the medium comprises at least 60% N-2-hydroxyethylpiperazine-N′-ethane-sulphonic acid-buffered Dulbecco's modified Eagle's medium, 5 to 15% sodium bicarbonate, 2 to 10% fetal bovine serum, 10 to 100 mg/ml gentamicin, 20 to 300 mg/ml vancomycin, and 0.1 to 5 mg/ml amphotericin B.

Conveniently, the medium has a volume between 10 and 100 ml.

Advantageously, the receptacle is made from a plastics material.

Conveniently, the penetrating element is a hypodermic needle.

Preferably, the kit further comprises a mesh suitable for the substrate to be located, preferably wherein the mesh is a polyester mesh.

According to another aspect of the present invention there is provided the use of a kit according to the invention for storing cells or tissue.

Conveniently, the use further comprises culturing the cells or tissue on a substrate.

Advantageously, the substrate is a planar or arcuate substrate.

Preferably, a cell explant is located on the substrate.

Advantageously, the limbal epithelial transplant is stored at a temperature of between 3° C. and 37° C., preferably between 3° C. and 30° C., preferably between 18° C. and 28° C., more preferably between 20° C. and 25° C., more preferably between 22° C. and 24° C., more preferably 22° C. and 23° C., more preferably for a period of at least one, two, three or four days, more preferably for a period of at least seven days.

According to a further aspect of the present invention there is provided a method of storing cells or tissue comprising keeping the cells or tissue at a temperature of between 3° C. and 37° C. wherein the cells or tissue comprise limbal epithelial cells, conjunctival cells, corneal endothelial cells, retinal cells, mucosal cells, epidermal cells or bone marrow derived cells.

This permits storage of the cells with substantially no increase in the differentiation of the cells.

Preferably, the limbal epithelial cells, conjunctival cells, corneal endothelial cells, retinal cells, mucosal cells, epidermal cells or bone marrow derived cells are cultured cells.

According to another aspect of the present invention, there is provided a method of storing limbal epithelial cells, wherein there is substantially no increase in the differentiation of the cells.

Preferably, the limbal epithelial cells are cultured limbal epithelial cells.

In some embodiments, “substantially no increase in the differentiation of cells” means that there are at least 80%, 90%, 95% or 99% of the number of undifferentiated cells at the end of storage as at the beginning.

There are a number of tests that can be used to determine whether there is substantially no increase in the differentiation of cells. For instance, it may be determined by analysing various immunohistochemical markers in the cells. For example the markers p63, K19 and Vimentin can each be used to indicate that cells are undifferentiated. Another test for the absence of differentiation is that the cells are negative for expression of the marker K3. Another test is low or negative expression of the markers Cx43, K5, K14 and/or Integrin β1.

It is also to be noted that when the cells are cultured and differentiated, the medium in which they are located is generally changed every 2 to 3 days whereas in embodiments of the present invention such change of medium does not take place.

Conveniently, the method comprises keeping the cells or tissue at a temperature of between 3° C. and 37° C.

Preferably, the method further comprises the step of transporting the limbal epithelial cells between two locations.

Conveniently, the method comprises keeping the cells or tissue at a temperature of between 3° C. and 30° C., preferably between 18° C. and 28° C., preferably between 20° C. and 25° C., preferably between 22° C. and 24° C., preferably 22° C. or 23° C.

Preferably, the method comprises storing the cells or tissue submerged in a serum-free medium for a period of at least one day at a temperature of between 18° C. and 28° C.

Indeed, according to one specific aspect of the present invention, a method of storing cells or tissue comprising keeping the cells or tissue at a temperature of between 18° C. and 28° C. submerged in serum-free medium for a period of at least one day wherein the cells or tissue comprise limbal epithelial cells, conjunctival cells, corneal endothelial cells, retinal cells, mucosal cells, epidermal cells or bone marrow derived cells and are preferably cultured cells. This method permits storage of the cells with substantially no increase in the differentiation of the cells.

Advantageously, the method further comprises the step of locating the cells or tissue on a substrate, preferably a planar or arcuate substrate.

Preferably, the substrate is an amniotic membrane, a contact lens, a collagen gel or a plastics material.

Conveniently, the substrate is an amniotic membrane and the cells are epithelial cells and wherein the cells are located with the epithelial side facing the amniotic membrane.

Preferably, the amniotic membrane comprises an intact amniotic epithelium.

Advantageously, the method further comprises the step of attaching the substrate to a polyester mesh.

Preferably, the cells or tissue comprise limbal epithelial cells and have been obtained from a region of a donor's eye, the region comprising the sector 30° either side of the topmost position of the eye.

Advantageously, the region comprises the sector 15° either side of the topmost position of the eye.

Conveniently, the method comprises keeping the cells or tissue at the temperature for a period of at least one day, preferably at least two days, more preferably at least three days, more preferably at least four days, more preferably for a period of at least seven days.

Preferably, the cells or tissue are submerged within a liquid medium.

Advantageously, the liquid medium comprises minimal essential medium.

Conveniently, the liquid medium is a serum based medium.

Preferably, the liquid medium comprises fetal bovine serum.

Alternatively, the liquid medium is a serum-free medium.

Conveniently, the serum-free medium comprises: Optisol-GS or PAA-Quantum.

Alternatively, the serum-free medium comprises: a buffering agent and minimal essential medium.

Preferably, the buffering agent comprises HEPES (4-(2-hydroxyethyl)-1-piperazineethane-sulfonic acid), preferably at 25 mM concentration.

Advantageously, the minimal essential medium comprises amino acids, salts, glucose and vitamins.

Conveniently, the salts comprise at least one of potassium chloride, magnesium sulphate, sodium chloride and sodium dihydrogen phosphate and/or the vitamins comprise at least one of folic acid, nicotinamide, riboflavin and B-12.

Advantageously, the liquid medium comprises sodium bicarbonate.

Conveniently, the liquid medium comprises an antibiotic.

Preferably, the antibiotic is gentamicin, vancomycin, amphotericin B or mixtures thereof.

Advantageously, the liquid medium comprises at least 60% N-2-hydroxyethylpiperazine-N′-ethane-sulphonic acid-buffered Dulbecco's modified Eagle's medium, 5 to 15% sodium bicarbonate, 2 to 10% fetal bovine serum, 10 to 100 mg/ml gentamicin, 20 to 300 mg/ml vancomycin, and 0.1 to 5 mg/ml amphotericin B.

Conveniently, the liquid medium has a volume between 10 and 100 ml.

Advantageously, the method further comprises the step of adjusting the composition of the gas above the liquid medium.

Preferably, the cells or tissue are stored for a period of at least 3 days, more preferably at least 7 days, more preferably at least 2 weeks, more preferably at least 3 weeks.

Advantageously, the cells or tissue are stored in a closed system.

Conveniently, the method further comprises the step of culturing the cells or tissue, prior to storage.

Preferably, the step of culturing the cells or tissue comprises maintaining the cells or tissue under the following conditions: a temperature of between 35° C. and 39° C., preferably 37° C.; submerged in a liquid medium suitable for cell culturing under an atmosphere comprising between 90% and 99% oxygen and between 10% and 1% carbon dioxide, preferably 95% oxygen and 5% carbon dioxide.

Conveniently, the method uses the kit of the invention.

According to another aspect of the present invention, there is provided a method of providing limbal cell explants comprising removing limbal epithelial cells from a donor's eye only in the region comprising the sector 30° either side of the topmost position of the eye. In this aspect, the remaining sectors of the donor's eye remain in situ.

Preferably, the method comprises removing the limbal epithelial cells from the donor's eye only in the region comprising the sector 15° either side of the topmost position of the eye.

Conveniently, the donor is a cadaver.

According to a further aspect of the present invention, there is provided a cell storage medium comprising HEPES buffer and minimal essential medium at a concentration of between 20 mM and 30 mM, preferably 25 mM.

Preferably, the cell storage medium further comprises an antibiotic, preferably gentamicin.

In this specification, reference to “cells” includes reference to “tissues” comprising such cells.

In this specification, the term “cultured” is used in relation to cells and tissue to indicate that the cells or tissue have been subjected to “ex vivo expansion” and the terms can be used interchangeably. For example, in the case of a limbal cell explant, the tissue is removed from a donor at which point the tissue typically comprises only 2 or 3 stem cells (the remainder of the cells being differentiated cells). The explant is then subject to culturing at 37° C. in hormonal epithelial medium under an atmosphere of 95% oxygen and 5% carbon dioxide. The medium is replaced every second day. The process of culturing, or ex vivo expansion, results in an increase in the number of undifferentiated cells in the explant due to replication of the stem cells. Cultured cells generally lack the connective tissue of uncultured cells and comprise fewer cell layers. Cultured cells are more suitable for transplantation than tissue taken directly from a donor.

Limbal cells may be characterised by reference to certain cytoplasmic/nuclear and cell surface markers such as is described in Schlötzer-Schrehardt U. et al, Experimental Eye Research, Vol 81, 3, Sep. 2005, 247-264. Exemplary stem cell markers are provided in Table 1.

TABLE 1
Semiquantitative immunohistochemical localization of
stem cell markers in human ocular surface epithelia

| | Corneal | | Limbal | | Conjunctival | |
|---|---|---|---|---|---|---|
| | epithelium | | epithelium | | epithelium | |
| | | Supra- | | Supra- | | Supra- |
| Markers | Basal | basal | Basal | basal | Basal | basal |

Cytoplasmic/nuclear markers

| | | | | | | |
|---|---|---|---|---|---|---|
| Keratin | ++ | ++ | − | + | − | − |
| K3/K12 | | | | | | |
| Keratin | − or (+) | − | + | (+) | ++ | − |
| K5/K14 | | | | | | |
| Keratin K19 | − | − | ++ | − | (+) | ++ |
| Vimentin | − | − | ++ | (+) | − | − |
| α-enolase | (+) | − | ++ | (+) | ++ | − |
| Metallothionein | − | + | (+) | + | − | + |
| p63 | (+) | − | ++ | (+) | (+) | − |
| Nestin | ++ | ++ | − | − | − | − |

Cell surface markers

| | | | | | | |
|---|---|---|---|---|---|---|
| Connexin 43 | ++ | + | − | + | − | − |
| E-cadherin | ++ | ++ | (+) | ++ | (+) or + | ++ |
| P-cadherin | (+) | − | − or (+) | − | (+) | − |
| β-catenin | ++ | ++ | ++ | ++ | ++ | ++ |
| Integrin α2 | ++ | + | − or ++ | + | ++ | ++ |
| Integrin α3 | ++ | + | − or ++ | + | ++ | + |
| Integrin α6 | ++ | + | − or ++ | + | ++ | + |
| Integrin αν | ++ | + | ++ | + | ++ | + |
| Integrin β1 | ++ | + | ++ | + | + | + |
| Integrin β2 | + | + | + | + | + | + |
| Integrin β4 | ++ | + | − or ++ | + | ++ | + |
| Integrin β5 | + | − | + | (+) | + | − |
| Integrin α3β1 | ++ | + | − or ++ | + | ++ | + |
| EGF-R | ++ | + | ++ | + | ++ | + |
| KGF-R bek | − | − | (+) | − | − | − |
| HGF-R met | (+) | − | (+) | − | − | − |
| NGF-R TrkA | + | (+) | + | − | − or (+) | − |
| Transferrin-R | − or (+) | + | − or (+) | + | − or (+) | + |
| CD71 | | | | | | |
| TGF-β-RI | ++ | + | ++ | + | ++ | (+) |
| TGF-β-RII | ++ | + | ++ | + | ++ | − |
| ABCG2 | − | − | ++ | − | − | − |

−, undetectable; (+), weak positivity; +, moderate positivity; ++, strong positivity.

### BRIEF DESCRIPTION OF THE DRAWINGS

Embodiments of the invention will now be described, by way of example only, with reference to the accompanying figures which show the following.

FIG. 1 is a cross-sectional view of an amniotic membrane storage device in accordance with one embodiment of the present invention.

FIG. 2 is a cross-sectional view of an amniotic membrane storage device in accordance with the second embodiment of the present invention.

FIG. 3 is a perspective view of the amniotic membrane storage device of the second embodiment.

FIG. 4 is a perspective view of the amniotic membrane storage device of a third embodiment.

FIG. 5 is a perspective view of a the amniotic membrane storage device of a fourth embodiment with hidden detail shown.

FIG. 6 shows images of organ culture preservation of cultured epithelium. FIG. 6A is an image of one embodiment in which a graft is attached to a polyester membrane carrier. The membrane carrier is attached to a rubber stopper of a glass infusion bottle that contains a storage medium. FIG. 6B is an image of cultured epithelium fully immersed in organ culture medium. The amniotic membrane was fastened to the polyester membrane carrier at four corners using a 6-0 monofilament suture. AM=amniotic membrane. E=limbal explant. PM=polyester membrane.

FIG. 7 shows images of stained sections on non-preserved epithelium and epithelium after one week storage at 23° C. (original magnification ×100). FIGS. 7A and 7B show staining with haematoxylin and eosin. FIGS. 7C and 7D show immunostaining of p63. FIGS. 7E and 7F show immunostaining of K19. FIGS. 7G and 7H show immunostaining of Vimentin.

FIG. 8 is a diagram of eye bank storage of cultured human limbal epithelial cells (HLEC). FIG. 8A shows the removal of limbal tissue from an eye. The limbal explant is excised from the healthy eye (for autologous transplantation) or a cadaver eye (for allogenic transplantation). FIG. 8B shows HLEC cultures. HLEC 31 are cultured for three weeks on a human amniotic membrane 32 that is fastened using a suture to a polyester membrane 33 of a culture plate insert. FIG. 8C shows eye-bank storage of cultured HLEC. The polyester mesh membrane with the cultured HLEC attached, is stored for one week at 23° C. in organ culture medium consisting of Dulbecco's modified Eagle's medium with 7.5% sodium bicarbonate, 8% fetal bovine serum, 50 μg/ml gentamicin, 100 μg/ml vancomycin and 2.5 μg/ml amphotericin B. This provides the benefits of i) flexibility in scheduling transplantation; ii) time to perform microbiological testing; and iii) safe tissue transportation.

FIG. 9 shows images of sections stained with haematoxylin and eosin in cultured human limbal epithelial cells after three weeks' culture (A) and one week's storage at 31° C. (B) and 5° C. (C). The arrowheads denote detachment of epithelial cells, whereas the arrows show basal layer detachment from the amniotic membrane. Original magnification: ×400.

FIG. 10 shows images of transmission electron micrographs showing cultured human limbal epithelial cells after three weeks' culture and one week's storage at three different temperatures. (A) 3-week HLEC cultures demonstrated a multilayered epithelium with numerous intercellular desmosomes (B, arrows) and hemidesmosomes (C, arrows) promoting adhesion to the amniotic membrane. (D) Under organ culture conditions at 31° C., dilated intercellular spaces, detachment of desmosome complexes (arrows, inset), and poor adhesion to the amniotic membrane were revealed. (E) The original epithelial structure was preserved after one week of organ culture storage at 23° C. with numerous desmosomes (F, arrows) and hemidesmosomes (G, arrows). (H) Optisol-GS storage at 5° C. induced dilated intercellular spaces, detachment of epithelial cells, detachment of the epithelia from the amniotic membrane, and increased number of intracellular vacuoles. In addition to weak to moderate chromatin condensation (arrows), rupture of cell membranes (arrows) and dissolution of organelles (arrows) were regularly observed. Lc: Limbal epithelial cell; Am: Amniotic membrane; D: Desmosomes; Hd: Hemidesmosomes; Cc: Chromatin condensation; Rcm: Rupture of cell membranes; Do: Dissolution of organelles. Scale bars: 10 μm (A); 1 μm (B, C, F, G); 1 μm; 2 μm (D); 5 μm (E, H).

FIG. 11 shows images of sections of cultured human limbal epithelial cells following immunostaining of p63 (A, B, C), K19 (D, E, F), vimentin (G, H, I), and K3 (J, K, L) after three weeks' culture and one week's storage at 31° C. and 5° C. The expression of markers of undifferentiated cells (p63/K19/Vimentin) was maintained after 31° C. OC storage and hypothermic preservation. The undifferentiated nature of the cells following eye bank storage was supported by the negative expression of K3, a marker of corneal epithelial differentiation. Original magnification: ×400.

FIG. 12 is a histogram illustrating H&E apoptotic index, caspase-3 labeling index, and TUNEL labeling index in cultured human limbal epithelial cells after three weeks' culture and one week's storage at three different temperatures. Results are expressed as mean percent of the apoptotic or labeling index in the individual experimental groups. Error bars denote 1 SE.

FIG. 13 shows haematoxylin and eosin (HE) staining, cleaved caspase-3 immunohistochemistry, and TUNEL staining of cultured human limbal epithelial cells after one week's organ culture storage at 23° C. (A) H&E staining demonstrating an apoptotic epithelial cell with circular nuclear fragments (arrow). (B) Cleaved caspase-3-positive surface cells with cytoplasmic immunoreactivity and well defined nuclear membranes (arrowheads). (C) TUNEL positive surface cell (arrowhead). Original magnification: ×400.

FIG. 14 is a fluorescent image of CAM/EH-1 stained cultured human limbal epithelial cells after storage for 2 days in Optisol-GS at ambient temperature.

FIG. 15 is a fluorescent image of CAM/EH-1 stained cultured human limbal epithelial cells after storage for 4 days in Optisol-GS at ambient temperature.

FIG. 16 is a fluorescent image of CAM/EH-1 stained cultured human limbal epithelial cells after storage for 2 days in MEM+HEPES at ambient temperature.

FIG. 17 is a fluorescent image of CAM/EH-1 stained cultured human limbal epithelial cells after storage for 4 days in MEM+HEPES at ambient temperature.

FIG. 18 is a fluorescent image of CAM/EH-1 stained cultured human limbal epithelial cells after storage for 2 days in PAA-Quantum at ambient temperature.

FIG. 19 is a fluorescent image of CAM/EH-1 stained cultured human limbal epithelial cells after storage for 4 days in PAA-Quantum at ambient temperature.

FIG. 20 is a fluorescent image of CAM/EH-1 stained cultured human limbal epithelial cells after storage for 2 days in Cnt-20 at ambient temperature.

FIG. 21 is a fluorescent image of CAM/EH-1 stained cultured human limbal epithelial cells after storage for 4 days in Cnt-20 at ambient temperature.

FIG. 22 is an image of H&E stained 13-days cultured human limbal epithelial cells.

FIG. 23 is an image of H&E stained cultured human limbal epithelial cells after storage for 2 days in Optisol-GS at ambient temperature.

FIG. 24 is an image of H&E stained cultured human limbal epithelial cells after storage for 4 days in Optisol-GS at ambient temperature.

FIG. 25 is an image of H&E stained cultured human limbal epithelial cells after storage for 2 days in MEM+HEPES at ambient temperature.

FIG. 26 is an image of H&E stained cultured human limbal epithelial cells after storage for 4 days in MEM+HEPES at ambient temperature.

FIG. 27 is an image of H&E stained cultured human limbal epithelial cells after storage for 2 days in EpiLife at ambient temperature.

FIG. 28 is an image of H&E stained cultured human limbal epithelial cells after storage for 4 days in EpiLife at ambient temperature.

FIG. 29 is an image of H&E stained cultured human limbal epithelial cells after storage for 2 days in Cnt-20 at ambient temperature.

FIG. 30 is an image of H&E stained cultured human limbal epithelial cells after storage for 4 days in Cnt-20 at ambient temperature.

FIG. 31 is an image of H&E stained cultured human limbal epithelial cells after storage for 2 days in PAA-Quantum at ambient temperature.

FIG. 32 is an image of H&E stained cultured human limbal epithelial cells after storage for 4 days in PAA-Quantum at ambient temperature.

FIG. 33 is an image of H&E stained cultured human limbal epithelial cells, following staining with deltaNp63α antibodies, after storage for 2 days in Optisol-GS at ambient temperature.

FIG. 34 is an image of H&E stained cultured human limbal epithelial cells, following staining with deltaNp63α antibodies, after storage for 4 days in Optisol-GS at ambient temperature.

FIG. 35 is an image of H&E stained cultured human limbal epithelial cells, following staining with deltaNp63α antibodies, after storage for 2 days in PAA-Quantum at ambient temperature.

FIG. 36 is an image of H&E stained cultured human limbal epithelial cells, following staining with deltaNp63α antibodies, after storage for 4 days in PAA-Quantum at ambient temperature.

FIG. 37 is an image of H&E stained cultured human limbal epithelial cells, following staining with p63 antibodies, after storage for 2 days in Optisol-GS at ambient temperature.

FIG. 38 is an image of H&E stained cultured human limbal epithelial cells, following staining with p63 antibodies, after storage for 4 days in Optisol-GS at ambient temperature.

FIG. 39 is an image of H&E stained cultured human limbal epithelial cells, following staining with p63 antibodies, after storage for 2 days in PAA-Quantum at ambient temperature.

FIG. 40 is an image of H&E stained cultured human limbal epithelial cells, following staining with p63 antibodies, after storage for 4 days in PAA-Quantum at ambient temperature.

FIG. 41 is an image of H&E stained cultured human limbal epithelial cells, following staining with Keratin 19 antibodies, after storage for 2 days in Optisol-GS at ambient temperature.

FIG. 42 is an image of H&E stained cultured human limbal epithelial cells, following staining with Keratin 19 antibodies, after storage for 4 days in Optisol-GS at ambient temperature.

FIG. 43 is an image of H&E stained cultured human limbal epithelial cells, following staining with Keratin 19 antibodies, after storage for 2 days in PAA-Quantum at ambient temperature.

FIG. 44 is an image of H&E stained cultured human limbal epithelial cells, following staining with Keratin 19 antibodies, after storage for 4 days in PAA-Quantum at ambient temperature.

FIG. 45 is an image of H&E stained cultured human limbal epithelial cells, following staining with Keratin 3 antibodies, after storage for 2 days in Optisol-GS at ambient temperature.

FIG. 46 is an image of H&E stained cultured human limbal epithelial cells, following staining with Keratin 3 antibodies, after storage for 4 days in Optisol-GS at ambient temperature.

FIG. 47 is an image of H&E stained cultured human limbal epithelial cells, following staining with Keratin 3 antibodies, after storage for 2 days in FAA-Quantum at ambient temperature.

FIG. 48 is an image of H&E stained cultured human limbal epithelial cells, following staining with Keratin 3 antibodies, after storage for 4 days in PAA-Quantum at ambient temperature.

FIG. 49 shows images of viability stained cultured human limbal epithelial cells following 2-week storage and 3-week storage and a positive control.

FIG. 50 shows images of H&E stained cultured HLECs after 2 and 3 weeks storage at 23° C. following additional immunohistochemical staining.

FIG. 51 shows images of cultured HLECs following 1 week storage at 23° C. on intact (A, C & E) and denuded (B, D & F) amniotic membrane.

FIG. 52 shows images of cultured HLECs following 1 week storage at 23° C. on intact (A, C & E) and denuded (B, D & F) amniotic membrane.

FIG. 53 shows a diagram of the experimental design of Example 8.

FIG. 54 shows images of sections stained with haematoxylin and eosin in cultured human limbal epithelial cells of superior, nasal, inferior, and temporal limbal origin. S: superior; N: nasal; I: inferior; T: temporal; 1: donor 1; 2: donor 2; 3: donor 3; 4: donor 4; R: right; L: left. Original magnification: ×400.

FIG. 55 is a graph showing a comparison of average (±SEM) number of cell layers in cultured human limbal epithelial cells of superior, nasal, inferior, and temporal limbal origin. */** Significant difference from the superior group.

FIG. 56 shows images of sections of cultured human limbal epithelial cells of superior, nasal, inferior, and temporal origin with immunostaining of p63, ΔNp63α, ABCG2, K19, Vimentin, Integrin β1, PCNA, Ki67, CK3, CK5, and E-Cadherin. No evidence of major phenotypical differences was found in cultured HLEC of different limbal origin. Original magnification: ×400.

### DETAILED DESCRIPTION

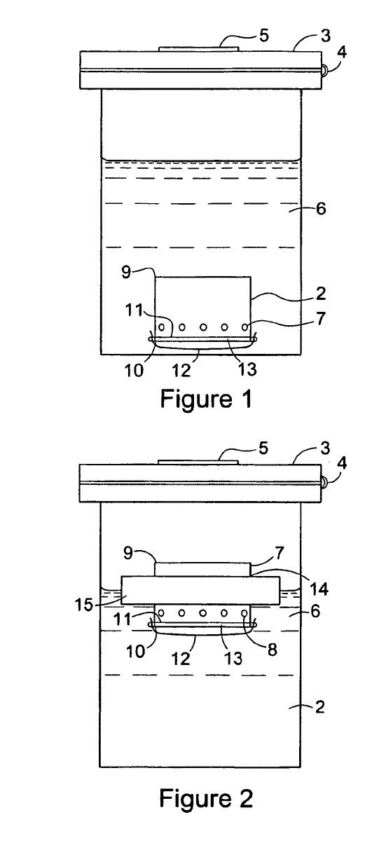

Referring to FIG. 1, an amniotic membrane storage device 1 comprises a cylindrical receptacle 2 made from a plastics material.

At the top of the receptacle 2 there is provided a cap 3, sealingly connected to the receptacle 2 via a hinge 4. The cap 3 is made from a rigid material (e.g. a plastics material) and is generally circular. In the centre of the cap 3, a circular section is not made from the rigid material but is instead replaced by a circular septum 5, made from a resilient material such as rubber. The septum 5 is such that it can be penetrated by, for example, a hypodermic needle and, when the hypodermic needle is removed, a seal is formed. Thus the septum 5 permits access to the interior of the receptacle in a sealed manner.

The receptacle 2 contains a liquid medium 6. In this embodiment, the medium is organ culture medium but in other embodiments, a different medium may be used such as CnT 20 medium. A serum-free medium such as Optisol GS, or PAA Quantum has certain advantages over of medium containing serum because the risk of infection being passed in the medium is eliminated. Furthermore, the contents of serum-free media can be replicated more accurately which is of significance when comparative studies are carried out. Another exemplary serum-free medium is 25 mM HEPES and MEM (Minimal Essential Medium) and 50 μg/ml gentamicin.

Also located in the receptacle 2, beneath the level of the medium 6, is a frame or culture insert 7. The frame 7 comprises a hollow cylinder. In the wall of the cylinder there are provided a series of apertures 8, equally spaced about the circumference, located around three-quarters away from the upper end 9 of the frame 7 and the lower end 10 of the frame 7. Also located in the cylindrical wall of the frame 7 is a circumferential furrow 11 which is located between the apertures and the lower end 10 of the frame 7.

In use, an amniotic membrane 12 is attached to the frame 7 as will now be described. The frame 7 is removed from the receptacle 2 and the amniotic membrane is stretched across the lower end 11 of the frame 7. Since the frame 7 is a hollow cylinder, the lower end 10 of the frame 7 forms a circular opening surrounded by a peripheral wall. A guide sleeve (not shown) is provided. The guide sleeve is a cylindrical rod of the same diameter as the frame 7. An elastic rubber band 13 is rolled on to one end of the guide sleeve and the guide sleeve is then lined up with the frame 7, the amniotic membrane 12 being located therebetween. The elastic band 13 is then rolled from the guide sleeve onto the frame 7 and lodges in the furrow 11 such that the outer edge of the amniotic membrane 12 is sandwiched between the rubber band 13 and the frame 7. The guide sleeve is then discarded. This means of attaching the membrane 12 to the frame 7 is relatively quick and easy.

It is to be noted that in variants of this embodiment, the guide sleeve is hollow and has an interior diameter slightly greater than the external diameter of the frame 7. In use the rubber band 13 is located on the guide sleeve and the guide sleeve is located over the frame. The rubber band 13 is then slipped off the end of the guide sleeve, directly on to the frame 7 in order hold the amniotic membrane in place.

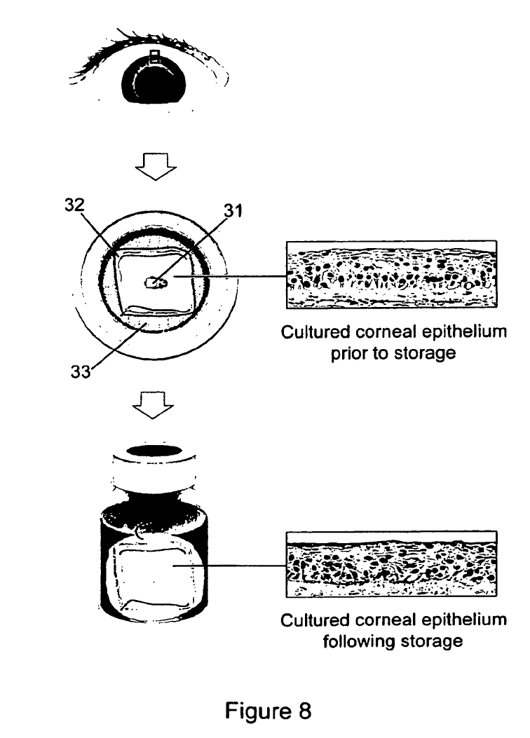

In the meantime, an explant is harvested from the limbus of either a patient (in the case of an autologous transplant) or a donor. More specifically, a limbal ring of tissue is made by means of two trephines punching a disc with a diameter of 15 mm. The disc comprises a section of cornea in addition to parts of the adjoining sclera. The central section of the explant is then subject to trephination to remove a circular section of approximately 7.5 mm in diameter to leave an annular ring of width of around 4 mm. The harvesting of the explant is known in the art. However, in this embodiment, a section from the ring is obtained from the so-called “12 o'clock position” or “superior position” that is to say the upper part of the cornea, as is shown in FIG. 8A, and this section is used for the subsequent procedure. The 12 o'clock position is the sector which exists 30° either side of the topmost position of the cornea or more preferably 15° either side of the topmost position. It has been found that explants obtained from this position have a higher proliferative potential and have a higher number of cell layers than explants from other positions which may provide greater mechanical strength. Indeed, the advantages of explants obtained from the superior position mean that explants can be obtained only from the superior position from donors (either living donors or cadavers) with tissue from other positions remaining in situ.

The explant section is then laid on the amniotic membrane 12 with the epithelial side of the explant facing the amniotic membrane.

The frame 7, together with the amniotic membrane 12 and the explant are then immersed in the medium 6 within the receptacle 2 and the cap 3 is sealed over the top of the receptacle 2. The apertures 8 permit the free movement of the medium 6 over the upper side of the membrane 12.

The receptacle 2 is stored at 22° C. or 23° C., in other words “room temperature”, which allows the explant to be stored and transported without any complicated and expensive cooling or warming equipment. Samples of the medium 6 are taken by insertion of a hypodermic needle through the septum 5. Usually, only one sample of medium need be taken for microbiological assessment but samples may be taken periodically if necessary. If required, the medium 6 can be substantially removed from the receptacle 2 and replaced with fresh medium, again by insertion of a needle via the septum 5. However, because of the sealed nature of the receptacle 2, there is little or no risk of contaminating of the medium 6 during such a process. The medium does not need to be changed during storage and there is no significant increase in differentiation of the cells during storage.

When the explant is required for implantation, the cap 3 is opened and the frame 7 removed from the medium 6, allowing free access to the cultured limbal epithelial cells.

In a variant to this embodiment, after the frame 7 is sealed within the receptacle 2, the concentration of the gases within the receptacle 2, above the level of the medium 6 is varied. This is achieved by inserting first and second needles through the septum 5 a short distance so that the needles remain above the level of the medium 6. Gas having a desired composition is inserted into the receptacle 2 via the first needle while an identical volume of gas is removed from the receptacle 2 via the second needle. In this way, the oxygen tension, for example, of the gases above the medium 6 may be adjusted.

In the above described embodiment of the invention, a rubber band 13 is provided to attach the amniotic membrane 12 to the frame 7. However, in other embodiments of the invention, the elastic band 13 is replaced with a suture, a cord (such as a metal cord) or some other elongate or annular resilient element. Furthermore, in some other embodiments, the membrane 12 is attached to the frame 7 by means of a ring clamp.

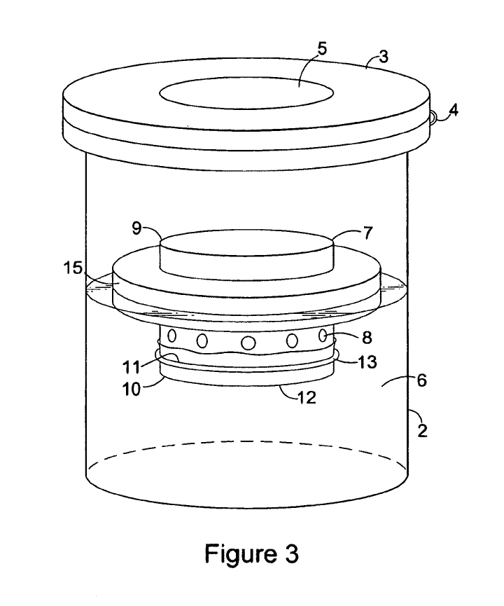

Referring now to FIGS. 2 and 3, a second embodiment of the present invention will be described in which like components have the same reference numerals as the first embodiment. In this embodiment, in addition to the circumferential furrow 11, a circumferential groove 14 is provided in the cylindrical frame 7, around three-quarters of the way from the lower end 10 to the upper end 9 of the frame 7. The groove 14 is significantly wider than the furrow 11. Located within the groove is an annular float 15 made from an expanded foam material. The inner diameter of the float 15 is sized so as to fit snugly in the groove 14. The outer diameter of the float 15 is sized so as to be slightly smaller than the inner diameter of the receptacle 12.

This second embodiment of the invention is used in the same way as the first embodiment except that, prior to insertion of the frame 7 into the receptacle 2, the float 15 is slid over the upper end 9 of the frame 7 and into the groove 14. When the frame 7 is located within the receptacle 2, the buoyancy of the float 15 causes the float 7 to be supported within the medium 6. Thus the membrane floats at a predetermined depth within the medium 6. It is to be noted that the upper end 9 of the frame 7 is located above the level of the medium 6. However, medium flows freely in and out of the apertures 8 so the upper side of the amniotic membrane 12 is exposed to the medium 6.

As in the first embodiment, the receptacle 2 is stored at 22° C. or 23° C. and samples of the medium 6 are taken by means of a hypodermic needle inserted by the septum 5. In this embodiment, it is necessary for the needle either to be inserted vertically downwardly from the septum 5 and thus to extract medium 6 from within the frame 7 or for the needle to be inserted at a significant angle to the vertical so that medium 6 can be obtained from the beyond the outer edge of the float 15.

It is to be appreciated that aside from providing the role of supporting the frame 7 within the medium 6, the float 15 also prevents the amniotic membrane 12 from touching the sides of the receptacle 2, while allowing the frame 7 (and the amniotic membrane) to rotate within the receptacle 2. Furthermore, the impact absorbing nature of the expanded foam material means that the foam 15 protects the frame and the amniotic membrane 12 from minor knocks and impacts to the receptacle 2.

It is also to be appreciated that, in alternative embodiments, variants to the float 15 are provided. For example, in some embodiments, the float 15 is made from an expanded polystyrene material. Furthermore, in some other embodiments, a sector of the annular float 15 is not present so as to allow easier insertion of a needle into the medium 6 at the position of the missing sector. In further embodiments, instead of providing a single annular float 15, a plurality of floats are provided about the circumference of the frame 7. For example, in one embodiment, three separate floats are provided spaced equally)(120° apart about the circumference of the cylindrical wall of the frame 7. The floats extend sufficiently radially outwardly of the frame 7 that they prevent the frame 7 from coming into contact with the receptacle 2 while the frame 7 is floating on the medium 6. In some embodiments, the float 15 is made from a material (such as foam material) which can be punctured by a hypodermic needle without losing buoyancy. In such embodiments, a sample of the medium 6 can be extracted by inserting a needle through the septum 5 and the float 15 in order to reach the medium 6.

In a further embodiment, the annular float 15 comprises an air or gas filled member. In further embodiments, a second annular ring is provided on the frame 7, located axially of the float 15, in the direction of the lower end 11 of the frame 7. The second ring is not buoyant but provides a protective role of preventing the lower end 11 of the frame 7 from touching the sides of the receptacle 2.

It is also to be understood that in further embodiments of the present invention the float 15 is omitted and a different means is provided for supporting the frame 7 within the medium 6. For example, in one embodiment, the frame 7 is suspended by means of a wire, attached to the frame 7 at one end and attached to the cap 3 at the other end. The length of the wire may be selected to be sufficiently short that the frame cannot come into contact with the walls of the receptacle 2 (unless the receptacle is put at an extreme angle). For this reason such an embodiment is particularly well suited to be combined with the features of the third embodiment infra which keeps the receptacle vertical irrespective of movement of the outer structure which contains it. Alternatively, a plurality of wires may be provided. In another embodiment, the float 15 is replaced with a plurality of legs which extend radially outwardly from the frame 7 and then descend below the lower end 11 of the frame 7. The legs then sit at the bottom of the receptacle 2 and the remainder of the frame 7 is supported at a predetermined level within the receptacle 2. Furthermore, the legs prevent the cylindrical wall of the frame 7 from touching the sides of the receptacle 2.

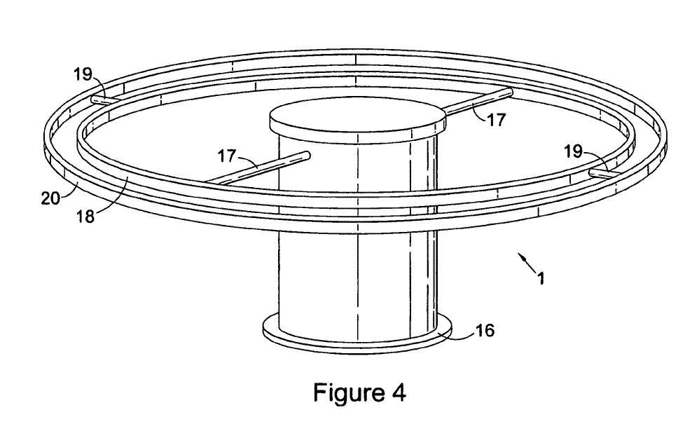

Referring to FIG. 4, a further embodiment of the present invention will now be described in which the amniotic membrane storage device is connected to a gimbal.

An amniotic membrane storage device 1 is provided as in the second embodiment and contains within the frame 7, float 15 etc. of the second embodiment. Also provided at the base of the receptacle 2 is a circumferential weight 16. Located at opposing sides of the exterior wall of the receptacle 2 and above the level of the centre of gravity of the receptacle 2 are provide two axles 17, extending radially outwardly from the receptacle 2. The first axles 17 are, in turn, connected to a first ring 18 which is located coaxially to and radially outwardly from the exterior of the receptacle 2. Located on the first ring 18 are two outwardly extending second axles 19 which are located 90° from the first axles 17 and are connected, in turn, to a second ring 20 which is located coaxially to and radially outwardly from the first ring 18. The second ring 20 is connected to an outer structure (not shown).

The receptacle 2 is rotatable relative to the first ring 18 about the axis defined by the first axles 17. In turn, the first ring 18 (and by virtue that its connection via the first axles 18 the receptacle 2) is rotatable relative to the second ring 20 about the axis defined by the second axles 19. Thus when the outer structure (which may, for example, be a storage box) is tilted, the receptacle 2 is able to swing within the first and second rings 18, 20 and, since the presence of the weight 16 results in the centre of gravity of the receptacle 2 being well below the first and second axles 17, 19, the receptacle 2 always swings so that the lower end thereof finds the lowest position and the receptacle is kept upright.

The advantage of this arrangement is that if the receptacle 2 (and the amniotic membrane and explant within it) are transported, for example by vehicle, movement of the outer structure will not affect the vertical orientation of the receptacle 2. Thus the amniotic membrane and the explant will remain supported at a predetermined depth of the medium 6.

In a variant of the third embodiment, a third ring is provided radially outwardly of the second ring 20. The second ring is connected to the third ring by third axles which allow rotation of the second ring relative to the third ring in an axis perpendicular to the axes defined by the first and second axles 17,19. The outer structure is connected to the third ring rather than the second ring. In this variant, any rotation of the outer structure along any axis is not transferred to the receptacle 2. Even a twisting motion of the outer structure about the vertical axis does not result in the receptacle 2 being rotated.

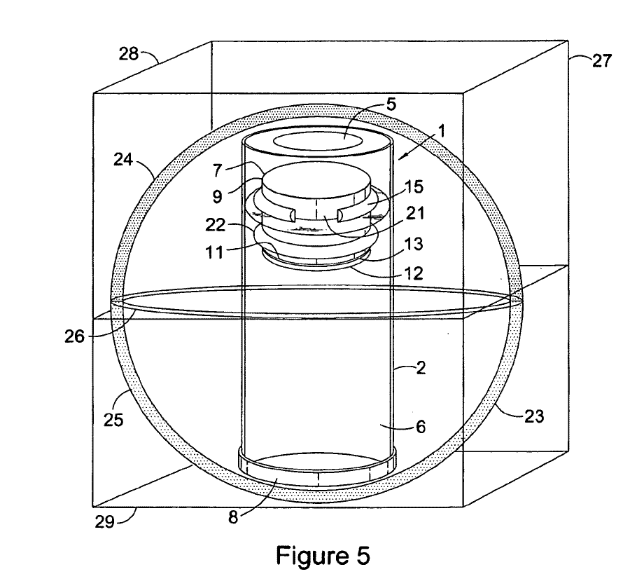

Referring now to FIG. 5, a fourth embodiment of the present invention will now be described. An amniotic membrane storage device 1 is provided in substantially the same form as the second embodiment. Thus there is a cylindrical frame 7 having an upper end 9 and a lower end 11. A series of apertures 8 is provided in the frame 7 as previously described. Adjacent the upper end 9 there is provided a groove for receiving a float 15 which fits circumferentially around the frame 7, except for a 60° C. sector 21 which permits access for a hypodermic needle to the side of the receptacle 7. At the lower end 11 of the frame 7 is located the amniotic membrane 12 which is held in place by a circumferentially located rubber band 13 which sits in a circumferential furrow adjacent the lower end 11. In this particular embodiment, a second groove is also provided for receiving a second annular ring 22 which lies between the float 15 and the rubber band 30, located circumferentially around the cylindrical frame 7. The annular ring 22 is not buoyant but acts to protect the frame 7 and prevent the frame 7 from contacting the inner wall of the receptacle 2. As in previous embodiments, the receptacle 2 contains a medium 6 in which the frame 7 is supported by the float 15. The receptacle 2 is sealed, the top having a septum 5 made from a resilient sheet, allowing access by a hypodermic needle to the interior of the receptacle. As in the third embodiment, a circumferential weight 16 is provided at the base of the receptacle 2.

The receptacle 2 is located within a spherical casing 23 which comprises upper and lower hemispheres 24, 25. The upper hemisphere 24 is red and the lower hemisphere 25 is green. The upper and lower hemispheres 24, 25 meet at an equator 26. The upper and lower hemispheres 24 and 25 have cylindrical recesses therein, which oppose each other when the upper and lower hemispheres 24, 25 are connected to form the casing 23 and which are sized to receive the receptacle 2. The upper and lower hemispheres 24, 25 have a screw thread for engaging one another at the equator 26. The inner casing 23 is located in an outer casing 27 which is in a shape of a cube and comprises an upper section 28 and a lower section 29, each forming half of the cube of the outer casing 27. The upper and lower sections 28, 29 each contain respective hemispherical recesses for receiving the inner casing 23. A fluid with oil-like properties is located between the inner casing 23 and the outer casing 27.

In use, the amniotic membrane and explant are attached to the frame 7 and located with the receptacle 2 as described above. The receptacle 2 is then placed in the cylindrical recess of the lower hemisphere 25, which can be identified due to its green colour. The upper hemisphere 24 is then located so that the receptacle 2 is received within the cylindrical insert therein. The upper and lower hemispheres 24, 25 are then connected to each other by the screw thread. The inner casing 23 is then located within the outer casing 27 and the oil-like fluid provided between the inner and outer casings 23, 27. Thereafter, the outer casing 27 can be transported and the amniotic membrane and explant are stored securely inside. More specifically, any impacts are absorbed by the float 15 and the annular ring 22 and a rotation of the outer casing 27 does not affect the orientation of the receptacle 2, since the inner casing 23 rotates, under the influence of gravity (especially on the weight 16), so that the receptacle 2 always remains upright. Consequently, the amniotic membrane 12 is kept at a predetermined depth of medium 6. Furthermore, any rotation of the outer casing 27 about a vertical axis will not generally result in any rotation of the inner casing 23 at all.

It is to be appreciated that a wide range of dimensions for the components of the fourth embodiment are possible but the following may be considered exemplary. The receptacle 2 has a diameter of between 18 and 12 cm, preferably 10 cm and a height of between 18 and 12 cm, preferably 10 cm. The frame 7 has a diameter of between 3 and 5 cm, preferably 4 cm. The cylindrical recesses in the upper and lower hemispheres 24, 27, are each 5.1 cm deep and have an inner diameter of 10.1 cm. Such an arrangement of dimensions permits the frame 7 to be located with the receptacle 2 and the receptacle 2 to be located within the inner casing 23.

In a variant to this embodiment, the annular ring 22 is replaced with a second float such that the frame 7 is supported higher within the medium 6 and the membrane 12 is supported at the gas-fluid interface, that is to say with around 1 or 2 mm of the medium 6 above the amniotic membrane 12. This results in so-called “air-lifting” of the amniotic membrane 12. Studies by Prunieras M et al. have indicated that skin cultures grown with air-lifting appear morphologically to be more similar to in vivo tissues than to tissues grown submerged beneath culture medium. Indeed, it is to be understood that the air-lifting technique is not limited to this embodiment of the invention and may be applied to the other embodiments as well.

It is to be appreciated that even if there is some evaporation of the medium 6, the membrane 12 is held at the same depth beneath the surface of the medium 6. Therefore, if the air lifting technique is used, as described above, the required depth of the membrane 12 is maintained, irrespective of any change in the amount of medium 6 in the receptacle 2 (e.g. following the removal of a sample for microbiological assessment).

While the above described embodiments of the invention involve the provision of an amniotic membrane 12 on the frame 7, it is to be understood that in alternative embodiments of the present invention a different planar substrate is used to culture the explant and the amniotic membrane may be replaced, for example, with a collagen gel or a plastic material. Alternatively, the substrate may be arcuate (i.e. have an arcuate cross section) such as, for example a contact lens.

It is also to be understood that while the above described embodiments involve the culturing and storage of a limbal cell explant, in alternative embodiments, explants of other types of cells are used. For example, in other embodiments, explants of conjunctival, endothelial, retina, mucosal, epidermal (i.e. skin) or bone marrow derived cells are used. In further.embodiments, tissue comprising such cells is stored.

It is, furthermore, to be noted that while the above described embodiments involved the explant method of culturing epithelial cells, the present invention is equally applicable to the cell suspension approach.

### EXAMPLES

### Example 1

This example relates to a study into the preservation of cultured limbal epithelial cells over extended periods of time.

### Materials and Methods

### Cell Culture and Organ Culture Preservation of Limbal Epithelial Cells

The research was conducted in accordance with the Declaration of Helsinki, and consent was obtained for the use of donor tissue for research purposes. Human amniotic membranes, preserved as reported previously,¹ were attached to the polyester membrane of Netwell culture plate inserts (Costar, Corning, New York, N.Y., USA) using 6-0 non-absorbable sutures. Eyes were enucleated from cadavers, and explant cultures (n=32) were prepared as described previously by Meller et al.²

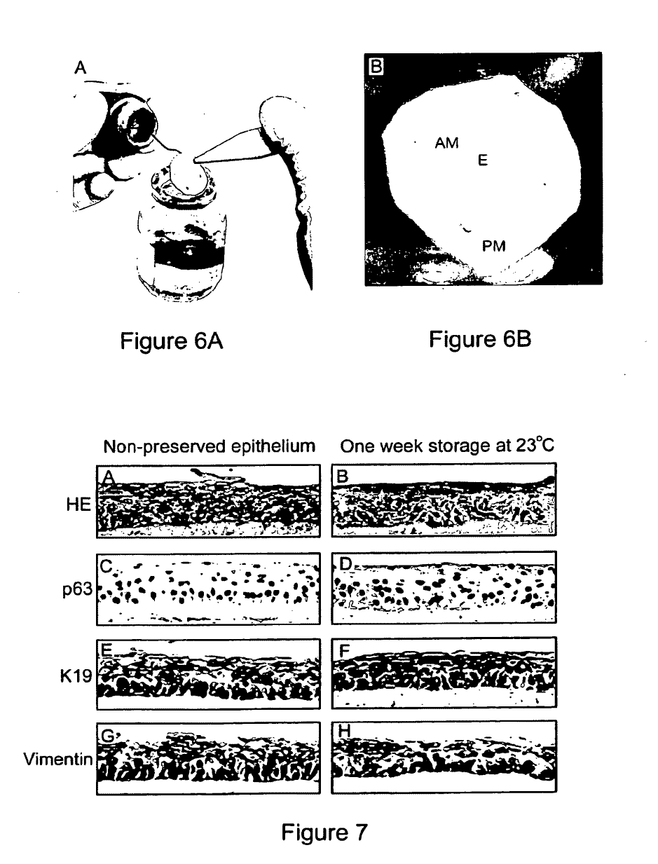

Limbal explants exposed to dispase (Roche Diagnostics, Basel, Switzerland) were incubated with the stromal side facing the amniotic membrane for 21 days at 37° C. in a medium consisting of N-2-hydroxyethylpiperazine-N′-ethane-sulphonic acid-buffered Dulbecco's modified Eagle's medium containing sodium bicarbonate and Ham's F12 (Sigma-Aldrich, St Louis, Mo., USA) supplemented with 5% fetal bovine serum, 0.5% dimethyl sulphoxide, 2 ng/ml human epidermal growth factor, 5 μg/ml insulin, 5 μg/ml transferrin, 5 ng/ml selenium, 3 ng/ml hydrocortisone, 30 ng/ml cholera toxin (Biomol, Exeter, UK), 50 μg/ml gentamicin and 1.25 μg/ml amphotericin B. The polyester mesh bottom with the cultured epithelium attached was released using a steel blade and suspended in a sterilised 50 ml glass infusion bottle using an Ethicon Ethilon 6-0 monofilament suture, which was tied to the edge of the polyester membrane (FIG. 6). The epithelia (n=16) were incubated for 1 week at 23° C. in an organ culture medium containing N-2-hydroxyethylpiperazine-N′-ethane-sulphonic acid-buffered Dulbecco's modified Eagle's medium with 7.5% sodium bicarbonate, 8% fetal bovine serum, 40 mg/ml gentamicin (Garamycin), 100 mg/ml vancomycin (Abbott Laboratories, Abbott Park, Ill., USA) and 1.5 mg/ml amphotericin B.

### Cell Viability Analysis

Mitochondrial function, an indicator of cell viability, was measured using a colorimetric assay, as reported previously.³⁻⁵ This technique is based on mitochondrial enzyme reduction of the water-soluble tetrazolium salt-8-(2(2-methoxy-4-nitrophenyl)-3-(4-nitrophenyl)-5-(2,4-disulphophenyl)-2H-tetrazolium monosodium salt) and spectrophotometric quantification of the water-soluble formazan dye generated. Initially, a calibration curve was created to investigate the relationship between the optical density and the number of viable cells in samples from non-preserved cultured epithelial cells. Disks (n=12) of cultured epithelium were trephinated using biopsy punches (Kai Industries, Gifu, Japan) of different diameters (2, 3, 4, 5 and 6 mm). They were then incubated in 20 μl CCK-8 solution (Alexis Corporation, Lausen, Switzerland) and 200 μl organ culture medium for 2 h. The solution was analysed colorimetrically at 450 nm in an automated microplate reader (Kinetic-QCL, Bio-Whittaker, Walkersville, Md., USA). The discs were subsequently trypsinised, and cell numbers were counted directly using the trypan blue dye exclusion technique. Based on measurements of 3 mm epithelial discs, the optical density after preservation (n=8) was calculated as the percentage of that before preservation (n=8).

### Light Microscopy and Immunohistochemistry

Preserved epithelia (n=8) and non-preserved epithelia (n=8) were fixed in neutral buffered 4% formaldehyde and embedded in paraffin. Serial sections of 5 μm thickness were routinely stained with haematoxylin and eosin. Immunohistochemistry was performed with a panel of antibodies (Table 2). To visualise the immunoreactions, we used a standard peroxidase technique (DAB detection kit) in a Ventana ES Immunohistochemistry Instrument (Tucson, Ariz., USA). Optimal antibody dilutions were determined by titration using the positive controls recommended by the manufacturers. The expression pattern was evaluated by two independent investigators.

### Statistical Analysis

Data are presented as mean (SD). SPSS V.14.0 was used to assess the cell viability (correlation analysis and t tests for two independent groups). A p value of 0.05 was considered significant.

### Results

### Viability

A linear relationship was observed between the optical density and the viable cell number in samples from non-preserved cultured epithelial cells (correlation r=0.97). The optical density of the non-preserved epithelia was 0.27 (0.03), whereas that of the preserved epithelia was 0.23 (0.05), giving a viability percentage of 84% (20%). No significant difference was found between the two groups (p).07).

### Light Microscopy and Immunohistochemistry

On the whole, the cell borders were maintained, and the nuclei showed no sign of degeneration (FIG. 7B). The epithelia attached well to the amniotic membrane. Mild intercellular oedema was occasionally observed. No change in staining pattern was revealed for K19, vimentin, K3, K5 and K14. Minimal changes were disclosed for Ki67, p63, Cx43, E-cadherin and integrin b-1 (Table 2, FIG. 7).

TABLE 2
Semiquantitative immunohistochemical characteristics of non-preserved cultured epithelium
and cultured epithelium preserved for one week at 23° C. (room temperature)

| | | | Non-preserved epithelium | | | One week storage at 23° C. | | |
|---|---|---|---|---|---|---|---|---|
| Specificity | Clone | Source, dilution | B | SB | S | B | SB | S |
| P63 | 4A4 | DAKO¹, ( 1/25) | 3 | 2 | 0-1 | 3 | 3 | 0-1 |
| Cytokeratin 19 | RCK108 | DAKO¹, ( 1/200) | 2 | 2 | 2 | 2 | 2 | 2 |
| Vimentin | VIM 3B4 | Ventana Medical Systems² | 3 | 2 | 1 | 3 | 2 | 1 |
| Ki67 | MIB-1 | DAKO¹, ( 1/75) | 1 | 0-1 | 0 | 0-1 | 0 | 0 |
| Cytokeratin 3 | AE5 | ImmuQuest³, ( 1/500) | 0 | 0 | 0 | 0 | 0 | 0 |
| Cytokeratin 5 | XM26 | Novocastra Lab. Ltd.⁴, ( 1/600) | 3 | 3 | 3 | 3 | 3 | 3 |
| Cytokeratin 14 | LL02 | Novocastra Lab. Ltd.⁴, ( 1/80) | 3 | 3 | 3 | 3 | 3 | 3 |
| Connexin 43 | (polyclonal) | Sigma-Aldrich⁵, ( 1/500) | 2 | 2 | 1 | 1 | 1 | 0 |
| E-cadherin | NCH-38 | Novocastra Lab. Ltd.⁴, ( 1/25) | 1 | 2 | 0-1 | 1 | 2 | 0 |
| Integrin beta-1 | 7F10 | Novocastra Lab. Ltd.⁴, ( 1/10) | 2 | 1 | 0-1 | 2 | 0 | 0 |

¹Glostrup, Denmark;
²Tucson, AZ, USA;
³Cleveland, UK;
⁴Newcastle, UK;
⁵St. Louis, MO, USA
B = basal layer; SB = suprabasal layer; S = superficial layer
0 = undetectable; 1 = weak positivity; 2 = moderate positivity; 3 = strong positivity

### Discussion

Past studies have examined the epithelial proliferative potential of organ cultured corneoscleral rims as a source of limbal epithelial cells.⁶⁻⁸ However, no previous reports have examined organ culture preservation of ex vivo expanded limbal epithelial cells. This example shows that cultured limbal epithelial cells can be preserved in organ culture medium at room temperature for 1 week, while maintaining the original layered structure and undifferentiated phenotype.

The initial challenge was to find a suitable carrier for the amniotic membrane. Ultimately, only polyester membrane culture plate inserts met all our requirements. The membranes (1) were able to withstand the tension of the sutures and keep the amniotic membrane distended; (2) were easily released from the culture plate insert; (3) fitted into the glass infusion bottle; and (4) were easy to detach from the amniotic membrane.

Organ culture preservation of donor corneas is currently the most widely used corneal storage method in Europe,⁹ and the medium supplies the nutrients needed to maintain cellular metabolism in the tissue.¹⁰ The present example was conducted at room temperature (23° C.), which eliminated the need for heating cabinets and made it easier to distribute the transplants between eye departments. A few reports have been published that consider the influence of room temperature (23-25° C.) on corneas stored in culture media such as McCarey-Kaufman medium, 12 K-Sol medium,¹² TC 199 medium¹³ and RPME 1640 organ culture meditun.¹⁴ However, in these studies, the corneal endothelium has been the main focus of attention.

The linear relationship observed between the optical density and the cell number is consistent with the results of Kito et al,⁵ who reported a high correlation (R²=0.976). The results of the cell viability assay and the light microscopy examination indicated that the majority of the cultured epithelial cells were viable after preservation. Mild intercellular oedema occurred occasionally, which has previously been reported after organ culture storage.^{15,16}

As there were no data available for direct comparison with our immunohistochemical findings, we compared our data with the results of a study by Joseph et al,⁷ which investigated limbal explants stored in organ culture medium for 3-4 weeks. The expression of p63, vimentin, Ki67 and Cx43 was close to their results. However, in their study, only a few cells were positive for K19, and K3 was expressed in the superficial layer.

There is no standardised method of culturing limbal epithelial cells. In the present example, we used intact amniotic membrane without 3T3 fibroblast feeder layers or air-lifting, because previous studies have suggested that this method preserves the characteristics of limbal epithelial stem cells.^{2, 17, 18} However, the use of air-lifting has been reported to provide the epithelial sheet with increased mechanical strength,¹⁹ which may be beneficial prior to preservation in organ culture.

In conclusion, our study demonstrates that organ culture may preserve cultured epithelia for transplantation.

### Example 2

PURPOSE. Example 1 has described eye bank storage of cultured human limbal epithelial cells (HLEC) to provide a reliable source of tissue for treating limbal stem cell deficiency. The present study aimed at investigating whether conventional organ culture (OC) storage and Optisol-GS storage were applicable to cultured HLEC. We hypothesized that OC storage at 31° C., which is the preferred temperature in 26 out of 43 European Eye Banks (European Eye Bank Association Directory, 2007), and Optisol-GS hypothermic storage may preserve the characteristics of cultured HLEC. Accordingly, we compared these conventional storage methods with the novel storage method. Moreover, because cell death due to apoptosis has been reported in human corneal epithelium after OC culture storage⁵⁴ and hypothermic storage,^{55, 56} we studied expression of apoptosis-regulatory genes and examined apoptosis markers in cultured HLEC following eye bank storage.

METHODS. 3-week HLEC cultures were either organ-cultured at 31° C. or 23° C. or stored in Optisol-GS at 5° C. in a closed container for one week. Morphology was studied by light microscopy and transmission electron microscopy, and phenotypic characterization was assessed by immunohistochemistry. Apoptosis was evaluated by real-time RT-PCR microarray analysis, caspase-3 immunohistochemistry, and terminal deoxynucleotidyl transferase-mediated dUTP nick-end labelling (TUNEL).

RESULTS. The ultrastructure was preserved at 23° C., while storage at 31° C. and 5° C. was associated with enlarged intercellular spaces, separation of desmosomes, and detachment of epithelial cells. Cultured HLEC remained undifferentiated under all storage conditions. Expression of the anti-apoptotic gene BCL2 was prominently upregulated under storage at 23° C. and 5° C. Downregulation of BCL2A1, BIRC1, and TNF and upregulation of CARD6 under 23° C. and 5° C. storage conditions suggested a reduction in nuclear factor-KB activity. No significant increase in cleaved caspase-3 and TUNEL staining was observed in response to eye bank storage, and the labeling indices of cleaved caspase-3 (range 0.0%-4.7%) and TUNEL (range 0.0%-7.8%) were low.

CONCLUSIONS. These data indicate that OC storage of cultured HLEC at ambient temperature is superior to OC storage at 31° C. and Optisol-GS storage at 5° C., and that apoptosis is minimal following eye bank storage of cultured HLEC.

### Materials and Methods

Dulbecco's minimal essential medium (DMEM), HEPES-buffered DMEM containing sodium bicarbonate and Ham's F12 (1:1), Dulbecco's modified Eagle's medium, Hanks' balanced salt solution, fetal bovine serum (FBS), insulin-transferrin-sodium selenite media supplement, human epidermal growth factor, dimethyl sulfoxide, hydrocortisone, gentamicin, amphotericin B, and rabbit polyclonal anti-connexin 43 antibodies were purchased from Sigma-Aldrich (St. Louis, Mo.). Dispase II was obtained from Roche Diagnostics (Basel, Switzerland), cholera toxin A subunit from Biomol (Exeter, UK), Ethicon Ethilon 6-0 C-2 monofilament suture from Johnson & Johnson (New Brunswick, N.J.), Netwell culture plate inserts from Costar Corning (New York, N.Y.), vancomycin from Abbott Laboratories (Abbott Park, Ill.), Optisol-GS from Bausch & Lomb (Irvine, Calif.), and glass containers from OneMed (Vantaa, Finland). Mouse anti-p63 antibody (clone 4A4), mouse anti-CK19 antibody (clone RCK108), and mouse anti-Ki67 antibody (clone MIB-1) were obtained from Dako (Glostrup, Denmark), while mouse anti-vimentin antibody (clone VIM 3B4) was purchased from Ventana Medical Systems (Tucson, Ariz.) and mouse anti-CK3 antibody (clone AE5) from ImmuQuest (Cleveland, UK). The following were sourced from Novocastra Laboratories Ltd (Newcastle, UK): mouse anti-CK5 antibody (clone XM26), mouse anti-CK14 antibody (clone LL02), mouse anti-E-cadherin antibody (clone NCH-38), and mouse anti-integrin β1 antibody (clone 7F10). Rabbit polyclonal anti-caspase-3 antibody came from Cell Signaling Technology (Danvers, Mass.). Epon was purchased from Electron Microscopy Sciences (Hatfield, Pa.). The ArrayGrade FFPE RNA isolation kit, RT² Profiler Apoptosis PCR array (cat. no. APHS-012), True Labeling Picoamp kit, RT² PCR array first strand synthesis kit, and RT² Real-Time™ SYBR Green PCR master mix PA-012 were obtained from SuperArray Bioscience (Frederick, Md.). The 7900HT 384-well block used was purchased from Applied Biosciences (Foster City, Calif.), while the Colorimetric TUNEL System kit used was from Promega Corporation (Madison, Wis.).

### Human Tissue Preparation

Human tissue was handled according to the Declaration of Helsinki. Corneoscleral tissues were obtained from the Norwegian Corneal Eye Bank (Oslo, Norway) after the central corneal button had been used for corneal transplantation. The experiment was conducted using four pairs of corneoscleral rims from the same human donors as Example 1 and the study of the four experimental groups (3-week HLEC culture and storage at 31° C., 23° C., and 5° C.) was run concurrently. The limbal tissue was prepared as previously reported by Meller et al.²⁹ The tissue was rinsed three times with DMEM containing 50 μg/mL gentamicin and 1.25 μg/mL amphotericin B. After careful elimination of excessive sclera, conjunctiva, iris, and corneal endothelium, the remaining tissue was placed in a culture dish and exposed for 10 minutes to Dispase II (1.2 U/mL) in Mg²⁺ and Ca²⁺ free Hanks' balanced salt solution at 37° C. under humidified 5% carbon dioxide. Following one rinse with DMEM containing 10% FBS, every corneoscleral rim was divided into 12 limbal explants which were equally distributed between the four experimental groups.

### Human Limbal Explant Cultures on Intact Amniotic Membranes

Human AM were preserved in accordance with a method previously reported by Lee & Tseng⁵⁷ and according to the Declaration of Helsinki. After thawing at room temperature, AM with the epithelium intact and facing up was fastened to the polyester membrane of a culture plate insert using Ethicon Ethilon 6-0 monofilament sutures (FIG. 8), as previously reported in Example 1. Limbal explant cultures were prepared as previously described.²⁹ On the center of each AM insert, a human limbal explant was cultured in supplemented hormonal epithelial medium made of HEPES-buffered DMEM containing sodium bicarbonate and Ham's F12 (1:1). The medium was supplemented with 5% FBS, 0.5% dimethyl sulfoxide, 2 ng/mL human EGF, 5 μg/mL insulin, 5 μg/mL transferrin, 5 ng/mL selenium, 3 ng/mL hydrocortisone, 30 ng/mL cholera toxin, 50 μg/mL gentamicin, and 1.25 μg/mL amphotericin B. Cultures were incubated for 3 weeks at 37° C. in an atmosphere of humidified 5% carbon dioxide and 95% air, and the medium was changed every 2 to 3 days.

### Eye Bank Storage of Cultured Human Limbal Epithelial Cells

The HLEC cultures (n=36) were prepared for eye bank storage as reported in Example 1, and 3-week HLEC cultures (n=12) served as controls. The polyester mesh membrane with the cultured epithelium attached was released using a steel blade and suspended in a sterilised 50-mL glass container using an Ethicon Ethilon 6-0 monofilament suture, which was tied to the edge of the polyester membrane and the rubber cap (FIG. 8). The cultured HLEC were stored for 1 week in either 50 mL organ culture medium containing Dulbecco's modified Eagle's medium with 7.5% sodium bicarbonate, 8% FBS, 50 μg/mL gentamicin, 100 μg/mL vancomycin, and 2.5 μg/mL amphotericin B at 31° C. (n=12) and 23° C. (n=12), or in 50 mL Optisol-GS at 5° C. (n=12). The glass containers were each closed by a rubber cap to establish a closed tissue storage system.

### Histology and Immunostaining

Eight cultures from each experimental group were fixed in neutral buffered 4% formaldehyde and embedded in paraffin. Serial sections of 5 μm were routinely stained with haematoxylin and eosin (H&E). Immunohistochemistry was performed with a panel of antibodies for markers of human ocular surface epithelia (Table 3). To visualize the immunoreactions, we used a standard peroxidase technique (DAB detection kit) in a Ventana ES Immunohistochemistry Instrument (Tucson, Ariz.). Optimal antibody dilutions were determined by titration using the positive controls recommended by the manufacturers. A conventional immunohistochemical scoring system was used as previously reported.^{58,59} The immunoreactivity was graded as 0 (undetectable), +(weak positivity of >50% cells), ++(intermediate positivity of >50% cells), +++(strong positivity of >50% cells). All scores were assigned at a magnification of ×400 by two independent experienced investigators blinded to the origin of the samples.

### Transmission Electron Microscopy

Four cultures from each experimental group were fixed in 2% glutaraldehyde in 0.2 M cacodylate buffer adjusted to pH 7.4, postfixed in 1% osmium tetroxide and dehydrated through a graded series of ethanol up to 100%. The tissue blocks were immersed in propylene oxide twice for 20 minutes and embedded in Epon. Ultrathin sections were cut on a Leica Ultracut Ultramicrotome UCT (Leica, Wetzlar, Germany) and examined using a Philips CM120 transmission electron microscope (Philips, Amsterdam, the Netherlands).

### Real-Time Quantitative RT-PCR

RNA was isolated from the formalin-fixed paraffin-embedded (FFPE) tissue applying the ArrayGrade FFPE RNA isolation kit, according to manufacturers protocol. Three biological replicates were randomly selected from each experimental group. The RT² Profiler human Apoptosis PCR Array was used to analyze mRNA levels of 84 key genes involved in apoptosis, in a 384-well format, according to the manufacturer's instructions. In brief, approximately 30-40 ng RNA was first amplified using a modified version of the True Labeling Picoamp kit. First-strand cDNA was synthesized using 400 ng of amplified cRNA using the RT² PCR array first strand synthesis kit C-02. This kit uses PowerScript reverse transcriptase and a combination of random primers and oligo dT primers. The total volume of the reaction was 20 μL, diluted to 100 μL. PCR reactions were performed using the Applied Biosystems 7900HT 384-well block using RT² Real-Time™ SYBR Green PCR master mix PA-012. The total volume of the PCR reaction was 20 μL. An equivalent of 0.4 ng of RNA was applied to the PCR reaction. The thermocycler parameters were 95° C. for 10 min, followed by 40 cycles of 95° C. for 15 s and 60° C. for 1 min. Gene expression of stored HLEC was compared with 3-week HLEC cultures. Relative changes in gene expression were calculated using the ΔΔCₜ (cycle threshold) method.⁶⁰ An average of the cycle numbers of the five housekeeping genes, GAPDH, Actin-β, β2m, Hprt1, and Rpl³d, was used to normalize the expression between samples. The expression data is presented as actual fold changes.

### Cleaved Caspase-3 Immunohistochemistry and TUNEL Assays

Immunohistochemistry was performed as described above with an antibody specific for cleaved caspase-3 (dilution 1:100). Terminal deoxynucleotidyl transferase-mediated dUTP nick-end labelling (TUNEL) was carried out using a Colorimetric TUNEL System according to the manufacturer's protocol. At ×400 magnification, cells from the whole length of the epithelial outgrowth with condensed nuclei and positively labeling with anti-caspase-3 and TUNEL were counted as apoptotic by two independent experienced investigators. The apoptotic index, caspase-3 labeling index, and TUNEL labeling index, were used as quantitative measures of apoptosis in histological sections as previously reported by Duan et al.⁶¹

### Statistical Analysis

Statistical comparison of real-time PCR data was performed with the non-paired Student's t-test (Excel, Microsoft, Redmond, Wash.) with 3-week HLEC cultures serving as controls.

The apoptotic and labeling indices were tested against the respective indices in 3-week HLEC cultures using the Mann-Whitney test (SPSS V.14.0, SPSS Inc., Chicago, Ill.). A p-value of <0.05 was considered significant.

### Results

### Epithelial Morphology

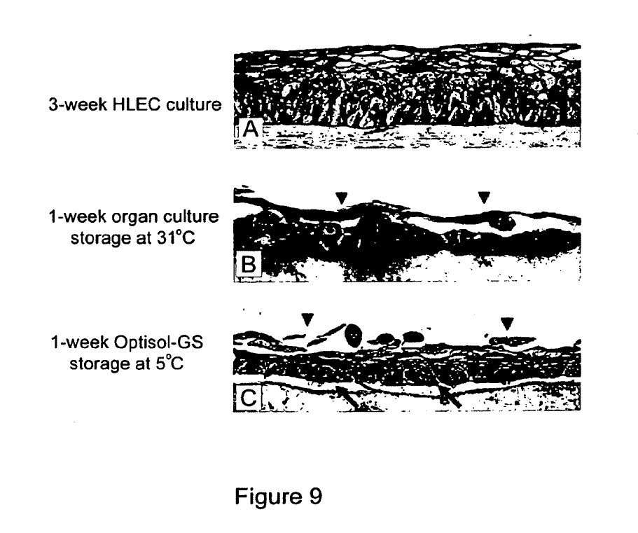

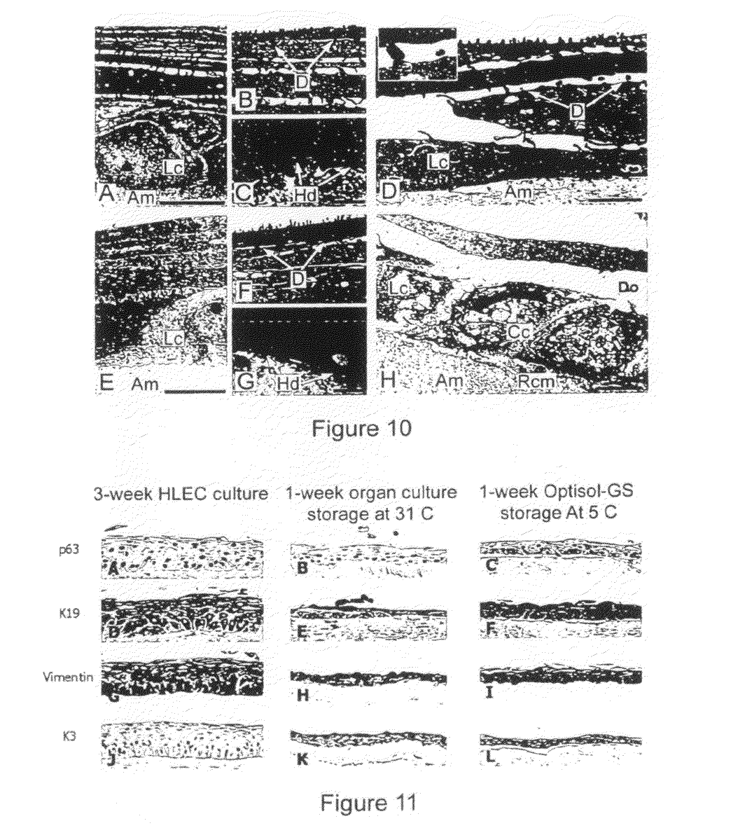

Following storage at 31° C., extensive detachment of epithelial cells was seen (FIG. 9B). Fibroblasts were noted in three out of eight replicates. Weak chromatin condensation was occasionally seen, but clumping of nuclear chromatin and rupture of the cell membranes were not observed. The spaces between adjacent cells increased considerably (FIG. 10D). Few desmosomes, separation of desmosomes, and detachment of desmosome complexes were revealed. The basal cells were poorly attached to the amniotic membrane via a small number of hemidesmosomes. Intracellular vacuoles were common.

Storage of HLEC cultures at 23° C. did not induce chromatin condensation, nuclear fragmentation, or clumping of nuclear chromatin, and cell membranes remained intact (FIG. 10E). Intercellular spaces increased slightly, and numerous desmosomal junctions were seen between adjacent superficial epithelial cells (FIG. 10F). The polymorphic basal cells attached well to the amniotic basement membrane by hemidesmosomes (FIG. 10G). Intracellular vacuoles were observed infrequently.

Storage of HLEC cultures in hypothermic conditions demonstrated considerable enlargement of intercellular spaces, separation of desmosomes, detachment of epithelial cells, detachment of the epithelia from the AM, and increased number of intracellular vacuoles (FIGS. 9C, 10H). In addition to weak to moderate chromatin condensation, rupture of cell membranes and dissolution of organelles were regularly observed.

3-week HLEC cultures served as controls and showed a multilayered epithelium (FIG. 10A) with numerous intercellular desmosomes (FIG. 10B) and hemidesmosomes (FIG. 10C).

### Phenotypic Characterization

The cultured HLEC remained undifferentiated (p63/K19/Vimentin positive and K3 negative) under 31° C. OC storage and hypothermic storage conditions (Table 3, FIG. 11).

### Apoptosis Gene Expression Profiling

Table 4 shows the anti- and pro-apoptotic genes in cultured HLEC following 1-week's storage at three different temperatures. Expression of DNA fragmentation factor (DFFA) was not significantly altered in stored HLEC, while the expression of caspase-3 was below the level of detection. Following storage at 23° C. and 5° C., upregulation of BCL2, downregulation of BCL2A1 and BIRC1, and reduced expression of TNF receptor signaling components (TNF and TRADD) were revealed. Furthermore, upregulation of components of the Fas-mediated pathway (FAS, FASLG, and FADD) and BAG4 and downregulation of PYCARD were observed under 23° C. storage conditions. Under all storage conditions, expression of BNIP2 was upregulated, whereas MCL1 expression was down-regulated.

### Quantification of Apoptotic Cells

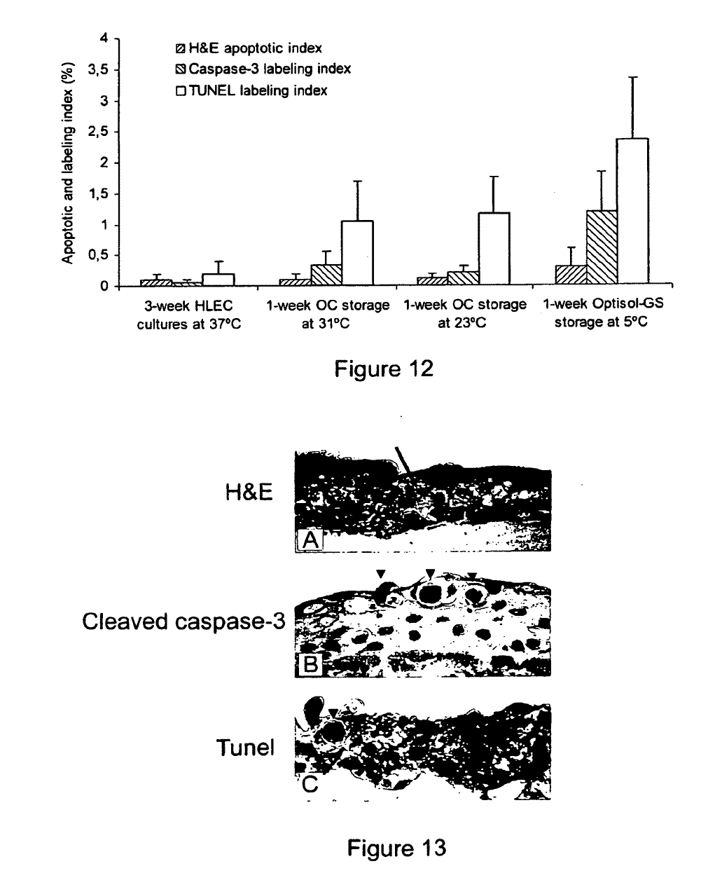

Few apoptotic cells were observed under all storage conditions (Table 5, FIGS. 12, 13) giving low labeling indices of caspase-3 (range 0.0% to 4.7%) and TUNEL (range 0.0% to 7.8%). When comparing the experimental groups with the control group, there was a trend towards higher apoptotic index with decreased storage temperature, although the differences were not statistically significant.

Discussion

In this example, conventional OC storage at 31° C. and hypothermic eye bank culture were clearly inferior in preserving the original layered structure of cultured HLEC compared with the 23° C. OC preservation method. Eye bank storage of cultured HLEC was associated with minor phenotypic changes and limited cell death due to apoptosis under all three storage conditions.

Interestingly, detachment of epithelial cells was observed consistently after storage at 31° C. and 5° C., in sharp contrast to storage at 23° C., where there was no sign of detachment. The morphological characteristics of cultured HLEC stored at 31° C. are in line with studies of organ cultured cornea performed at 31° C., which describe epithelial sloughing of two to three cell layers after 7 days⁶² and intracellular vacuoles.^{62, 63} Reduction of epithelial thickness has also been registered after OC storage of corneas at 37° C. ^{64, 65} and 34° C.⁵⁴ Furthermore, studies of organ cultured corneas at 37° C. have reported dilated intercellular spaces^{64, 65} and a decreased number of desmosomes,⁶⁴ both of which are consistent with our findings. With regard to storage at 5° C., morphological findings similar to those in the present study, were found in a study of human corneas stored for 6-10 days in Optisol-GS, demonstrating pronounced intracellular edema and separation of the cells below the superficial layer.⁶⁶

While no specific marker for the limbal epithelial stem cell has been identified to date, the description of an undifferentiated limbal epithelial phenotype currently relies on the combination of positive expression of putative stem cell-associated markers and negative or low staining of differentiation-associated markers. In the present example, the transcription factor p63 and the cytoskeletal proteins K19 and Vimentin were expressed following all storage conditions. Previous studies have shown that p63 is expressed in corneal epithelial cells with high proliferative capacity, denoted transient amplifying cells (TACs).⁶⁷⁻⁶⁹ K19 and Vimentin are localized to the basal cells of the limbal epithelium and have been suggested as stem cell candidate markers,^{58, 70, 71} however, a later study demonstrated that K19 was also expressed by corneal epithelial cells.⁵⁹ The undifferentiated nature of the cells following eye bank storage was supported by the negative expression of K3, a marker of corneal epithelial differentiation.⁷²

The positive expression of the gap junction protein Cx43 in our study is consistent with a recent investigation by Chen et al. who reported that 60% of cultured HLEC expressed Cx43.⁷³ However, previous reports have demonstrated that Cx43 is expressed in the suprabasal layer of limbal epithelium and suggested that Cx43 expression represents differentiation of corneal TACs.^{59, 74, 75} Furthermore, Hernandez-Galindo et al. suggested that the coexpression of delta p63 (clone 4A4) and Cx43 in HLEC cultures may indicate early TACs.⁶⁸ Positive expression of the keratin pair K5/K14 and Integrin PI may also be indicative of TAC differentiation as limbal and corneal basal cells are shown to express these markers.^{59, 41, 76, 77}

The inununohistochemical analyses may also provide insight into cell survival following eye bank storage of cultured HLEC. Maintenance of high p63 expression and minimal changes in expression of Ki67, a proliferating cell nuclear marker, suggest that eye bank storage preserves the proliferative capacity of cultured HLEC. Furthermore, the expression of the transmembrane receptor E-cadherin was sustained in most groups and is previously reported to facilitate cell proliferation and survival.⁷⁸

Intercellular edema may give an explanation for the considerable cell detachment under 31° C. and 5° C. storage conditions. However, cell death due to apoptosis^{79, 80} has been reported in human corneal epithelium after OC storage⁵⁴ and hypothermic storage.^{55,56} In the present example, signs of chromatin condensation were revealed under 31° C. and 5° C. storage conditions. Accordingly, we postulated that apoptosis might contribute to the detachment of epithelial cells, however, immunohistochemistry for cleaved caspase-3 and TUNEL showed no significant increase in response to eye bank storage.

The multi-gene profiling revealed interesting alterations in gene expression in cultured HLEC following eye bank storage. Several of the changes in gene expression in cultured HLEC under 23° C. and 5° C. storage conditions suggested a reduction in nuclear factor-κB (NF-κB) activity, in as much as several apoptosis regulating genes that are NF-κB targets were reduced in their expression, including BCL2A1, BIRC1, TNF, and PYCARD. The TNF receptor adapter protein, TRADD, was also reduced, while expression of BAG4, an antagonist of TNF receptor signaling, was increased. Furthermore, expression of CARD6, a modulator of certain NF-κB activation pathways,⁸¹ was increased. NF-κB protein is one of the major transcription factors.^{82, 83} The activation of NF-κB leads to synthesis of proinflammatory cytokines, including TNF-α and IL-1β, which mediate inflammatory and immune responses,⁸⁴ and protects the cells from undergoing apoptosis.⁸⁵⁻⁸⁷

Components (FAS, FASLG, and FADD) of the extrinsic pathway for cell death and caspase activation⁸⁸⁻⁹⁰ were all prominently upregulated under 23° C. storage conditions. Furthermore, expression of MCL1, an anti-apoptotic gene belonging to the BCL2 family, was profoundly downregulated, and expression of the BCL2 antagonists BNIP2 and BNIP3L was increased. It remains to be determined why these changes in gene expression were not associated with increased apoptosis. Downstream blocks to Fas-mediated apoptosis, including upregulation of the NF-κB-inducible anti-apoptotic proteins, BAG4 and CARD6, may have neutralized the increased expression of Fas-pathway components. In addition, the intrinsic pathway for caspase activation^{91,92} may have been inhibited by the strong upregulation of the BCL2, an inhibitor of apoptosis acting upstream of the activation of caspase in mitochondrial and endoplasmic reticulum pathways for cell death.⁹³ In support of the final supposition, BCL2 is suggested to modulate apoptotic cell desquamation in the human corneal epithelium.⁹⁴

In conclusion, the data present herein indicate that OC storage of cultured HLEC at ambient temperature is superior to OC storage at 31° C. and Optisol-GS storage at 5° C., and that apoptosis is minimal following eye bank storage of cultured HLEC. This indicates that eye bank storage of cultured HLEC may provide a reliable source of tissue for treating limbal stem cell deficiency.

TABLE 3
Semiquantitative immunohistochemical localization of ocular surface markers
in cultured human limbal epithelial cells after three weeks' culture
at 37° C. and one week's storage at 31° C., 23° C., and 5° C.

| | | 3-week | | 1-week | | 1-week | | 1-week | |
|---|---|---|---|---|---|---|---|---|---|
| | | HLEC culture | | organ culture | | organ culture | | Optisol-GS | |
| | Antibody | at 37° C.* | | storage at 31° C.† | | storage at 23° C.* | | storage at 5° C. | |
| Antigen | dilution | B | SB | B | SB | B | SB | B | SB |
| p63 | 1:25 | +++ | ++ | +++ | +++ | +++ | +++ | +++ | +++ |
| K19 | 1:200 | ++ | ++ | ++ | ++ | ++ | ++ | +++ | +++ |
| Vimentin | RTU | +++ | ++ | +++ | ++ | +++ | ++ | +++ | ++ |
| Ki67 | 1:75 | + | 0/+ | + | + | 0/+ | 0 | 0 | 0/+ |
| K3 | 1:500 | 0 | 0 | 0 | 0 | 0 | 0 | 0 | 0 |
| K5 | 1:600 | +++ | +++ | +++ | +++ | +++ | +++ | +++ | +++ |
| K14 | 1:80 | +++ | +++ | ++ | ++ | +++ | +++ | ++ | +++ |
| Cx43 | 1:500 | ++ | ++ | ++ | ++ | + | + | + | + |
| E-Cadherin | 1:25 | + | ++ | + | ++ | + | ++ | ++ | + |
| Integrin β1 | 1:10 | ++ | + | + | 0 | ++ | 0 | +++ | ++ |

The immunoreactivity was graded as 0 (undetectable), + (weak positivity of >50% cells), ++ (intermediate positivity of >50% cells), +++ (strong positivity of >50% cells). All scores were assigned at a magnification of x400 by two independent experienced investigators blinded to the origin of the samples.
*The results for 3-week HLEC culture at 37° C. and 1-week organ culture storage at 23° C. are previously reported.²⁷
†One out of eight samples was excluded from the analysis due to extensive ingrowth of fibroblasts.
B: basal layer, SB: suprabasal layer; RTU: ready to use.

TABLE 4
Fold up- or down-regulation of anti- and pro-apoptotic genes in cultured human limbal
epithelial cells following 1-week's storage at three different temperatures

| | | | 1-week organ | 1-week organ | 1-week |
|---|---|---|---|---|---|
| | | NLM Gene | culture storage | culture storage | Optisol-GS storage |
| | Gene | Accession | at 31° C. | at 23° C. | at 5° C. |
| Groups | Symbol | No. | (fold change) | (fold change) | (fold change) |
| Anti- | BAG4 | NM004874 | 4.51 | 7.65* | 2.49 |
| apoptotic | BCL2 | NM000633 | 2.09 | 18.87* | 12.56* |
| genes | BCL2A1 | NM004049 | −4.14 | −6.13* | −4.98* |
| | BIRC1 | NM004536 | −3.62 | −27.47* | −16.64* |
| | BIRC6 | NM016252 | −1.86 | 5.20* | 2.35 |
| | BIRC8 | NM033341 | −6.69* | −8.94* | −7.04* |
| | BNIP2 | NM004330 | 4.70* | 11.61* | 11.58* |
| | CARD6 | NM032587 | 1.43 | 6.14* | 3.42* |
| | MCL1 | NM021960 | −8.96* | −32.41* | −22.96* |
| Pro- | ABL1 | NM005157 | 10.46* | 4.22 | 2.54 |
| apoptotic | APAF1 | NM001160 | 2.16 | 4.88* | 3.22 |
| genes | BAK1 | NM001188 | −7.44* | −4.39 | −7.50* |
| | BCL2L11 | NM006538 | 2.08 | 8.72* | −3.20 |
| | BNIP3L | NM004331 | 5.54 | 13.27* | 7.58* |
| | CARD4 | NM006092 | 3.64 | 5.00* | −1.16 |
| | CARD8 | NM014959 | 4.97 | 30.16* | 10.36* |
| | CASP5 | NM004347 | −1.68 | 17.81* | 11.53* |
| | CASP6 | NM032992 | 15.78* | 15.21* | 17.79* |
| | CASP9 | NM001229 | −4.05* | −1.27 | −2.03* |
| | CIDEB | NM014430 | 10.21* | 23.86* | 11.75* |
| | FADD | NM003824 | −3.37 | 16.72* | 10.80* |
| | FAS | NM000043 | 1.71 | 4.93* | 6.00 |
| | FASLG | NM000639 | 1.01 | 14.25* | 6.93 |
| | GADD45A | NM001924 | 1.08 | −12.23* | −8.85* |
| | HRK | NM003806 | −2.02 | −3.01* | −3.13* |
| | NOL3 | NM003946 | 1.15 | −3.86* | −4.51* |
| | PYCARD | NM013258 | −1.34 | −5.67* | −2.27* |
| | RIPK2 | NM003821 | −2.13 | 7.07 | 9.44* |
| | TNF | NM000594 | −3.17 | −18.24* | −14.30* |
| | TNFRSF9 | NM001561 | −4.67 | −4.09* | −5.07* |
| | TNFSF10 | NM003810 | −9.32 | −4.21* | −3.23 |
| | TP53 | NM000546 | 1.30 | −5.75* | −4.28* |
| | TRADD | NM003789 | −1.81 | −9.11* | −4.25* |

Gene expression of stored HLEC was compared with 3-week HLEC cultures. Relative changes in gene expression were calculated using the ΔΔC₁ (cycle threshold) method.⁴¹ An average of the cycle numbers of the five housekeeping genes, GAPDH, Actin-β, β2m, Hprt1, and Rp113d, was used to normalize the expression between samples. The expression data is presented as actual fold changes.
*Significant fold changes (p < 0.05) on the basis of reliable C₁ values.

TABLE 5
Apoptotic index (AI), caspase-3 labeling index (CLI), and TUNEL labeling
index (TLI) in cultured human limbal epithelial cells after three weeks'
culture and one week's storage at three different temperatures
H&E apoptotic index (%)*

| | | | | | Percentage of | |
|---|---|---|---|---|---|---|
| | | | Std. | | samples with | |
| Group | N | Mean | Deviation | Maximum | AI = 0 | P-value† |
| 3-weeks HLEC culture | 8 | 0.1 | 0.2 | 0.6 | 75.0 | — |
| 1-week OC storage at 31° C. | 7∥ | 0.1 | 0.3 | 0.7 | 85.7 | 0.87 |
| 1-week OC storage at 23° C. | 8 | 0.1 | 0.2 | 0.6 | 62.5 | 0.88 |
| 1-week Optisol-GS storage at 5° C. | 8 | 0.3 | 0.8 | 2.3 | 87.5 | 0.80 |

Caspase-3 labeling index (%)‡

| | | | | | Percentage of | |
|---|---|---|---|---|---|---|
| | | | Std. | | samples with | |
| Group | N | Mean | Deviation | Maximum | CLI = 0 | P-value† |
| 3-weeks HLEC culture | 8 | 0.1 | 0.2 | 0.5 | 87.5 | — |
| 1-week OC storage at 31° C. | 7∥ | 0.3 | 0.6 | 1.6 | 71.4 | 0.54 |
| 1-week OC storage at 23° C. | 8 | 0.2 | 0.3 | 0.9 | 50.0 | 0.28 |
| 1-week Optisol-GS storage at 5° C. | 8 | 1.2 | 1.8 | 4.7 | 50.0 | 0.20 |

TUNEL labeling index (%)§

| | | | | | Percentage of | |
|---|---|---|---|---|---|---|
| | | | Std. | | samples with | |
| Group | N | Mean | Deviation | Maximum | TLI = 0 | P-value† |
| 3-weeks HLEC culture | 8 | 0.2 | 0.6 | 1.6 | 87.5 | — |
| 1-week OC storage at 31° C. | 7∥ | 1.0 | 1.7 | 4.8 | 42.9 | 0.19 |
| 1-week OC storage at 23° C. | 8 | 1.2 | 1.6 | 3.7 | 50.0 | 0.20 |
| 1-week Optisol-GS storage at 5° C. | 8 | 2.3 | 2.8 | 7.8 | 37.5 | 0.08 |

*H&E apoptotic index (AI) = Number of apoptotic cells (condensed nuclei) × 100/Total number of nuclei.
†P-values were calculated by testing the labeling index of the individual experimental group against the labeling index of 3-week HLEC culture.
‡Caspase-3 labeling index (CLI) = Number of activated caspase-3-positivc cells × 100/Total number of nuclei.
§TUNEL labeling index (TLI) = Number of TUNEL-positivc cells × 100/Total number of nuclei.
∥One out of eight samples was excluded from the analysis due to extensive ingrowth of fibroblasts.
OC: organ culture.

### Example 3

This example relates to a study into the transportation of cultured limbal epithelial cells. More specifically, the study was into the mechanical strength of cultivated sheets of human limbal epithelial cells (HLEC) for transplantation, in which grafts were transported a short distance.

### Methods

HLEC cultured from limbal explants positioned epithelial side up and down for three weeks in Netwell 74 μm polyester culture plate inserts, were transferred to a transport vial containing 25 mL organ culture medium and transported by bicycle in a distance of 3 kilometres. At arrival, epithelial disks of 5 mm were punched out using a trephine, and the membrane potential in individual HLEC were measured using patch clamp technique.

### Results

The membrane potential was similar in cultured HLEC expanded from limbal explants positioned epithelial side up and down which is indicative of viable cultured HLEC with intact cell membranes.

### Conclusion

This example shows that cultured HLEC may be transported at least a short distance.

### Example 4

### Serum Free Storage of Cultured Human Limbal Epithelial Cells

The purpose of this example was to show the feasibility of short-time serum free storage of cultured human limbal epithelial cells (HLEC).

### Methods

3-week HLEC cultures on amniotic membranes attached to polyester culture plate inserts were stored in Optisol-GS (Bausch & Lomb, Irvine, Calif.) for 2, 4, and 7 days at 4° C. in a closed Plastiques Gosselin polypropylene container (Hazebrouck Cedex, France). Gene expression in cultured HLEC was determined using Affymetrix GeneChip Human 1.0 ST Array and laser confocal microscope and digital imaging were used to distinguish live (calcein-acetoxymethyl ester (CAM)-positive) from dead (ethidium homodimer 1 (EH-1)-positive) cells. 13-days HLEC cultures on amniotic membranes attached to polyester culture plate inserts were stored in Optisol-GS (Bausch & Lomb), HEPES-MEM added Gentamicin (Sigma-Aldrich/Invitrogen), Epilife Medium (Invitrogen), Cnt-20 (CELLnTEC Advanced Cell Systems AG), and PAA-Quantum (E.Pedersen & Sønn) for 2 and 4 days at 22° C. in closed Plastiques Gosselin polypropylene containers. The cultures were characterised by light microscopy and viability was analyzed by CAM/EH-1-assay.

### Human Tissue Preparation

Human tissue was handled according to the Declaration of Helsinki. Cadaveric human corneas with research consent were obtained from the Centro de Oftalmologia Barraquer (Barcelona, Spain). The limbal tissue was prepared as previously reported by Meller et al.² The tissue was rinsed three times with DMEM containing 50 μg/mL gentamicin and 1.25 μg/mL amphotericin B. After careful elimination of excessive sclera, conjunctiva, iris, and corneal endothelium, the remaining tissue was placed in a culture dish and exposed for 10 minutes to Dispase II in Mg²⁺ and Ca²⁺ free Hanks' balanced salt solution at 37° C. under humidified 5% carbon dioxide. Following one rinse with DMEM containing 10% FBS, the corneoscleral rim was divided into 12 limbal explants.

### Human Limbal Explant Cultures

Human amniotic membranes (AM) were preserved in accordance with a method previously reported by Lee & Tseng¹ and according to the Declaration of Helsinki. Immediately prior to use, the AM was thawed, washed three times with sterile phosphate buffer solution (Sigma-Aldrich). The amniotic membrane was fastened with the epithelial side facing up to the polyester membrane of a culture plate insert using monofilament sutures as previously reported.^{46,96} On the center of each AM insert, the explants were cultured in supplemented hormonal epithelial medium with the epithelial side facing down.⁹⁷ The medium was made of HEPES-buffered DMEM containing sodium bicarbonate and Ham's F12 (1:1) and was supplemented with 5% FBS, 0.5% dimethyl sulfoxide, 2 ng/m human epidermal growth factor, 5 μg/mL insulin, 5 μg/mL transferrin, 5 ng/mL selenium, 3 ng/mL hydrocortisone, 30 ng/mL cholera toxin, 50 μg/mL gentamycin, and 1.25 μg/mL amphotericin B. Cultures were incubated for 13 days (study at ambient temperature) or 21 days (hypothermic storage in Optisol-GS) at 37° C. in an atmosphere of humidified 5% carbon dioxide and 95% air, and the medium was changed every 2 to 3 days.

### Hypothermic Storage of Cultured Human Limbal Epithelial Cells in Optisol-GS

Preparation for eye bank storage was performed in a class II safety cabinet. 25 mL of Optisol-GS preheated at 31° C. was added to radiation sterilized 90 mL Plastiques Gosselin polypropylene containers (interior diameter 43 mm). 3-week HLEC cultures in polyester culture plate inserts were transferred by a disposable forceps to the storage containers. The hinged cap with septum was closed to establish a closed tissue storage system, and the containers were stored for 2 (n=12), 4 (n=12), and 7 days (n=12) at 4° C. Gene expression in cultured HLEC was determined using Affymetrix GeneChip Human 1.0 ST Array and laser confocal microscope and digital imaging were used to distinguish live (calcein-acetoxymethyl ester (CAM)-positive) from dead (ethidium homodimer 1 (EH-1)-positive) cells.

### Serum Free Storage of Cultured Human Limbal Epithelial Cells at Ambient Temperature

Preparation for eye bank storage was performed in a class II safety cabinet. 20 mL of preheated Optisol-GS (Bausch & Lomb), 25 mM HEPES (Sigma)-MEM (Invitrogen) added 50 μg/ml Gentamicin (Sigma), Epilife Medium supplemented with 0.06 mM Calcium (Invitrogen), Cnt-20 (CELLnTEC Advanced Cell Systems AG), and PAA-Quantum (E.Pedersen & Sønn) was added to radiation sterilized 90 mL Plastiques Gosselin polypropylene containers (interior diameter 43 mm). The HLEC cultures in polyester culture plate inserts were transferred by a disposable forceps to the storage containers. The hinged cap with septum was closed to establish a closed tissue storage system, and the containers were stored for 2 (n=2 in each of the study groups) and 4 (n=2 in each of the study groups) at controlled ambient temperature (22° C.) in a wine cabinet.

### RNA Isolation, Chip Hybridization, Signal Normalization, and Statistical Analysis

Following storage, the culture plate inserts were transferred from the culture plates to modelling wax plates, and discs of cultured epithelium adherent to the underlying polyester membrane were trephinated from 3-week HLEC cultures (n=6) and HLEC cultures stored for 2 days (n=6), 4 days (n=6), and 7 days (n=6) using a 5 mm biopsy punch and stored in cryo tubes at −80° C. until further use. Samples (n=2) of amniotic membranes were processed by the similar procedure to assess the RNA level in devitalised amniotic epithelium without cultured HLEC. The tissue discs were thawed and total RNA was extracted with QIAGEN RNeasy Micro Kit according to the manufacturer's protocol. 350 μl of RTL buffer with beta-mercaptoethanol was added to the disks in microcentrifuge tubes and subjected to vortexing for 2 minutes. No QIAshredder Spin Column or equivalents were used. RNA concentration and purity was determined through measurement of A260/A280 ratios with the Nano prop ND-1000 Spectrophotometer (Thermo Fisher Scientific, Wilmington, Del., USA). Confirmation of RNA quality was assessed by use of the Agilent 2100 Bioanalyzer and RNA 6000 Nano Assay (Agilent Technologies, Santa Clara, Calif., USA). All the RNA samples had high quality and showed no signs of DNA contamination or RNA degradation. RNA samples were immediately frozen and stored at −80° C.

100 ng of total RNA was subjected to the GeneChip HT One-Cycle cDNA Synthesis Kit and GeneChip HT IVT Labeling Kit following the manufacturer's recommended protocol for whole genome gene expression analysis (Affymetrix). Labelled and fragmented single stranded cRNA were hybridized to the GeneChip Human Gene 1.0 ST Arrays (28869 genes). The arrays were washed and stained using FS-450 fluidics station (Affymetrix). The signal intensities were detected by Hewlett Packard Gene Array Scanner 3000 7G (Hewlett Packard, Palo Alto, Calif., USA).

The scanned images were processed using GCOS 1.4 (Affymetrix). The CEL files were imported into ArrayAssist Advanced Software ver.5.5.1 (Lobion Informatics, La Jolla, Calif., USA) and normalized using the Exon IterPLIER algorithm to calculate relative signal values for each probe set. In addition, quantile normalization was performed and a variance stabilization factor of 16 was used.

The scanned images were processed using GCOS 1.4 (Affymetrix). The CEL files were imported into Expression Console (Affymetrix) and normalized to calculate relative signal values for each probe set. For expression comparisons of different groups, profiles were compared using t-tests without corrections for multiple testing (Excel, Microsoft, Redmond, Wash.). Gene lists were generated with the criteria of p<0.05.

### Live-dead Viability Assays of Cultured HLEC Following Eye Bank Storage

Viability staining was performed using a calcein-acetoxymethyl ester (CAM)/ethidium homodimer 1 (EH-1) assay. CAM is converted to calcein by intercellular esterases in live cells, whereas EH-1 binds to cellular DNA in damaged and membrane-compromised cells. In brief, HLEC cultures prior to and following eye bank storage were incubated in phosphate-buffered saline (PBS) containing 2 μM CAM and 2 μM EH-1 (23° C., 45 minutes) and washed with PBS. Epithelial discs were trephinated using a 6 mm Kai biopsy punch (Kai Industries, Gifu, Japan) and mounted on cover-slipped glass slides. Fluorescent images of the basal layer were photographed using an Axiovert 100 LSM 510 laser scanning confocal microscope (Carl Zeiss Micoscopy, Oberkochen, Germany). Calcein was excited with an argon laser at 488 nm and emission was measured at 505-530 nm. The ethidium homodimer 1 dye was excited at 543 nm, and emission was collected at >570 nm. The number of live cells (green fluorescence) and dead cells (red fluorescence) was counted per 2 fields at 40-fold magnification, and the percentage of live cells was calculated. HLEC cultures (n=2) served as positive control for live cells.

### Histology and Immunostaining of Cultured HLEC Following Eye Bank Storage

HLEC cultures after 2-days storage (n=2 in each of the experimental groups) and 4-days storage (n=2 in each of the experimental groups) were fixed in neutral buffered 4% formaldehyde and embedded in paraffin. Serial sections of 5 μm were routinely stained with haematoxylin and eosin (H&E). Serial 5 μm sections were immunostained with antibodies recognizing p63 (1:25 dilution), deltaNp63α antibody (1:200), K19 (1:200), and K3 (1:500). To visualize the immunoreactions, a standard peroxidase technique (DAB detection kit) was used in a Ventana ES Immunohistochemistry Instrument (Tucson, Ariz.). Optimal antibody dilutions were determined by titration using the positive controls recommended by the manufacturers.

### Results

Gene fold change values in cultured human limbal epithelial cells following two, four, and seven days eye bank storage in Optisol-GS at 4° C. are presented in Table 6. Few genes (<1%0 of 28869 tested) were differentially and significantly expressed following two, four, and seven days eye bank storage in Optisol-GS at 4° C. Several genes belonging to the histone cluster 1 gene family (HIST1H4D, HIST1H3F, HIST1H4B, HIST1H4K, HIST1H4C, HIST1H4J, and HIST1H2BB) were differentially and significantly expressed, mostly following four and seven days hypothermic storage in Optisol-GS. It is known that animal cells react to mitogenic or stress stimuli by rapid up-regulation of immediate-early (IE) genes and a parallel increase in characteristic modifications of core histones: chromatin changes, collectively termed the nucleosomal response. The expression of limbal stem cell, progenitor, proliferation, and differentiation marker genes was not significantly changed during storage (Table 7).

TABLE 6
Gene fold change values in cultured human limbal epithelial cells following
two, four, and seven days eye bank storage in Optisol-GS at 4° C.

| | Affymetrix | 2-days storage | 4-days storage | 7-days storage |
|---|---|---|---|---|
| Gene Symbol | Unique ID | (fold change) | (fold change) | (fold change) |
| HIST1H4D | NM003539 | 1.0 | 3.2* | 6.6* |
| HIST1H3F | NM021018 | 1.3 | 2.5* | 4.9* |
| HIST1H4B | NM003544 | 1.2 | 2.7* | 3.9* |
| HIST1H4K | NM003541 | 1.1 | 2.4* | 3.1* |
| HIST1H4C | NM003542 | 1.1 | 1.6* | 2.4* |
| HIST1H4J | NM021968 | 1.1 | 2.0* | 2.2* |
| HIST1H2BB | NM021062 | 1.4* | 1.7* | 2.1* |
| SNORD3B-1 | NM003271 | 1.4 | 2.1* | 2.8* |
| RNU5E | NR002754 | 1.3 | 1.7* | 3.1* |
| RNU4B1 | NR002759 | 1.4 | 1.6* | 2.2* |
| SCARNA6 | NR003006 | 1.4 | 1.6 | 2.1* |
| FMO1 | NM002021 | 1.2 | 1.2 | 2.0* |
| SLC27A2 | NM003645 | 1.2 | 2.1* | 1.0 |

The CEL files were imported into Expression Console (Affymetrix) and analysed using Excel (Microsoft, Redmond, WA). Gene expression of stored HLEC was compared with 3-week HLEC cultures. The expression data is presented as actual fold changes.
*Significant fold changes (p < 0.05). Fold change values ≧ 2 are bolded.

TABLE 7
Limbal stem cell, progenitor, proliferation, and differentiation
marker genes that were equally expressed in cultured HLECs following
storage in Optisol-GS at 4° C. (Fold change < 2)

| | | Affymetrix |
|---|---|---|
| Marker | Gene symbol | probe set ID |
| A-enolase* | ENO1 | 7912198 |
| Keratin K3* | KRT3 | 7963523 |
| Keratin K5* | KRT5 | 7963427 |
| Keratin K12* | KRT12 | 8015115 |
| Keratin K14* | KRT14 | 8015366 |
| Keratin K15† | KRT15 | 8015337 |
| Keratin K19* | KRT19 | 8015349 |
| Vimentin* | VIM | 7926368 |
| Nestin* | NES | 7921088 |
| p63* | TP73L | 8084766 |
| PCNA‡ | PCNA | 8064844 |
| Ki67‡ | MKI67 | 7937020 |
| ABCG2* | ABCG2 | 8101675 |
| Connexin 43* | GJA1 | 8113467 |
| E-cadherin* | CDH1 | 7996837 |
| P-cadherin† | CDH3 | 7996819 |
| EGF-R* | EGFR | 8132860 |
| HGF-R* | MET | 8135601 |
| Integrin α2* | ITGA2 | 8105267 |
| Integrin α3* | ITGA3 | 8008237 |
| Integrin α6* | ITGA6 | 8046380 |
| Integrin α9* | ITGA9 | 8078619 |
| Integrin αv* | ITGAV | 8046861 |
| Integrin β1* | ITGB1 | 7932966 |
| Integrin β2* | ITGB2 | 8070826 |
| Integrin β4* | ITGB4 | 8009951 |
| Integrin β5* | ITGB5 | 8090162 |
| KGF-R bek* | FGFR2 | 7936734 |
| TrkA* | NTRK1 | 7906244 |
| TGF-β-RI* | TGFB1 | 8037005 |
| TGF-β-RII* | TGFB2 | 7909789 |
| Transferrin-R | TFRC | 8093053 |
| CD71* | | |
| Wnt-4† | WNT4 | 7913547 |

*Schlotzer-Schrehardt and Kruse⁶
†Figueira et al.⁷
‡General proliferation markers

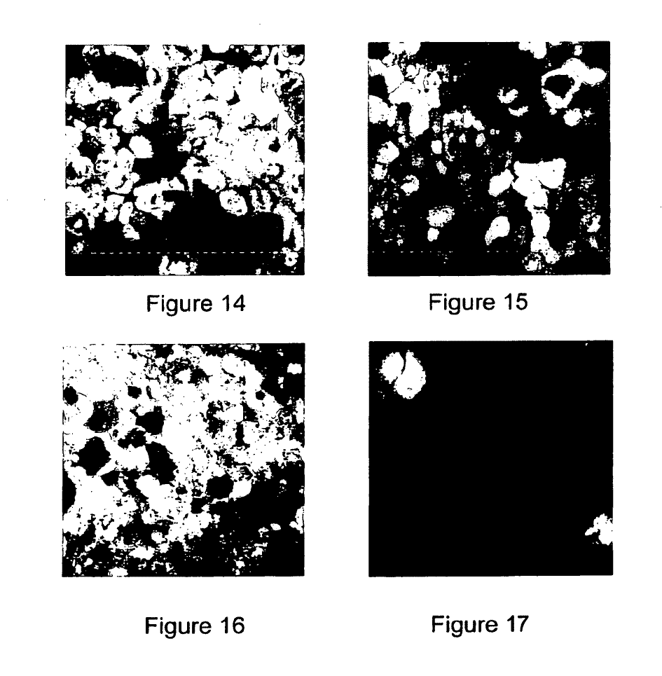

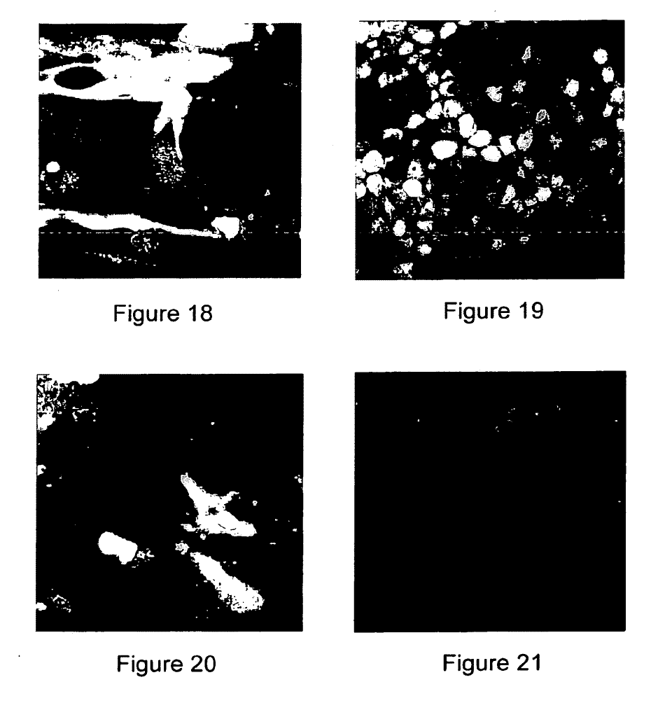

The laser confocal micrographs following serum free storage of cultured HLEC at ambient temperature are shown in FIGS. 14 to 21. The figures demonstrate viability staining of cultured HLEC after 2-days storage (FIGS. 14, 16, 18 and 20) and 4-days storage (FIGS. 15, 17, 19 and 21). Live cells are calcein AM positive and stain green, whereas ethidium homodimer 1 positive cells (dead) stain red. The results are summarised in Table 8.

TABLE 8

| | | |
|---|---|---|
| | Viability | Viability |
| | after storage | after storage |
| Media | for 2 days | for 4 days |

| | | | | |
|---|---|---|---|---|
| Optisol-GS | 99.1% | (105/106) | 99.6% | (282/283) |
| MEM + Hepes | 100% | (173/173) | 98.9% | (10/11) |
| PAA-Quantum | 88.5% | (20/23) | 99.1% | (567/572) |

| | | | |
|---|---|---|---|
| Cnt-20 | 100% | (37/37) | No data |

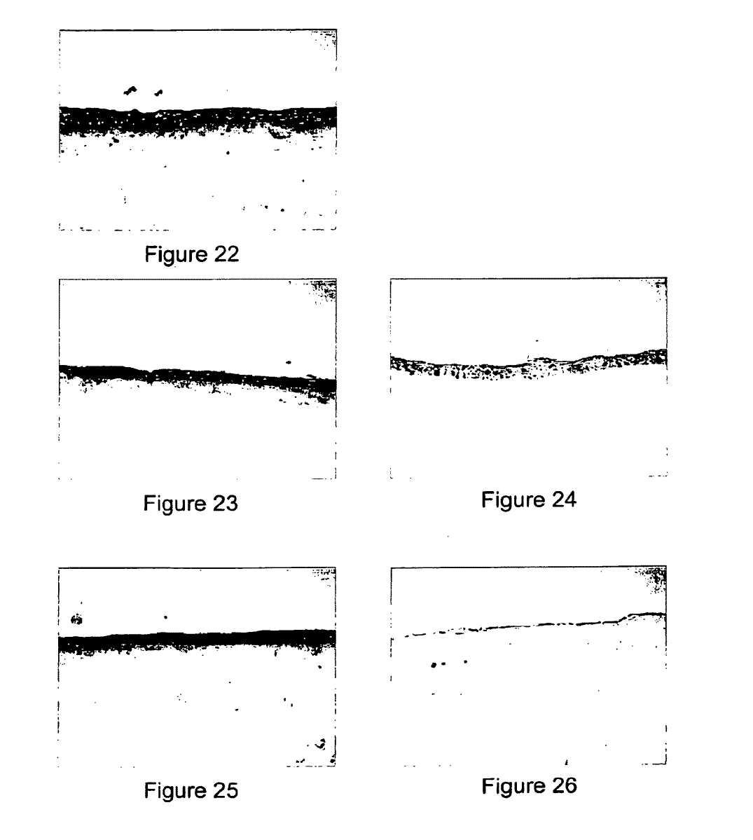

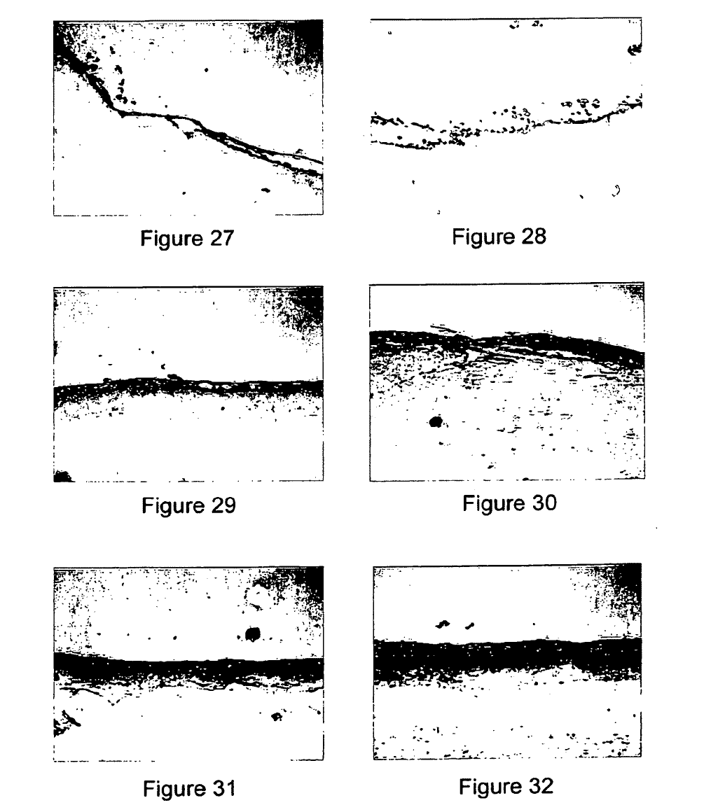

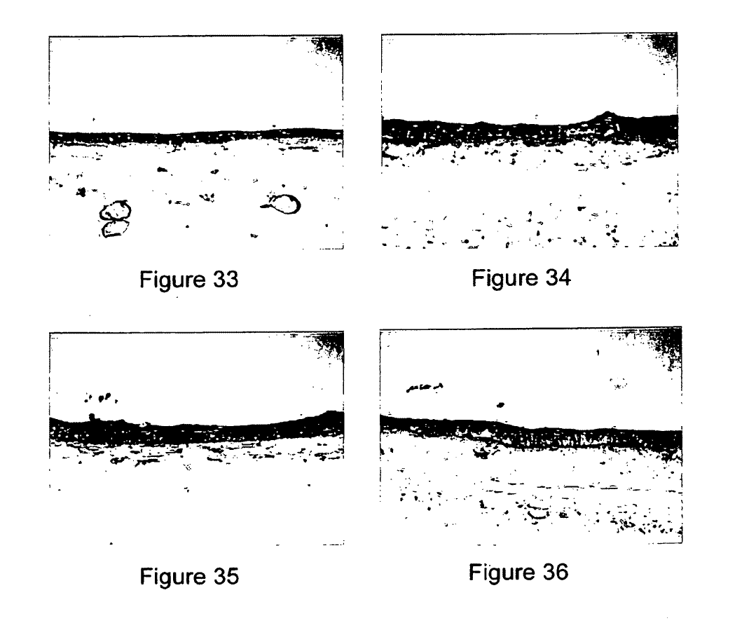

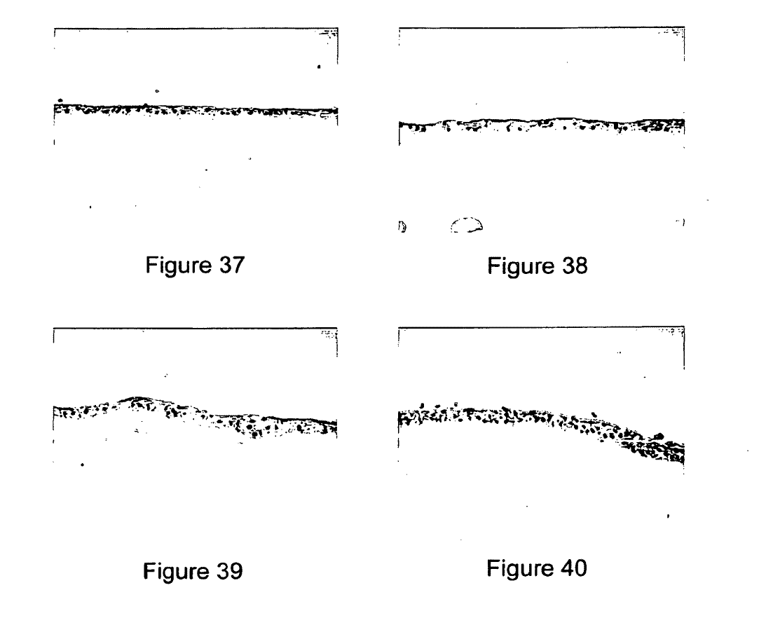

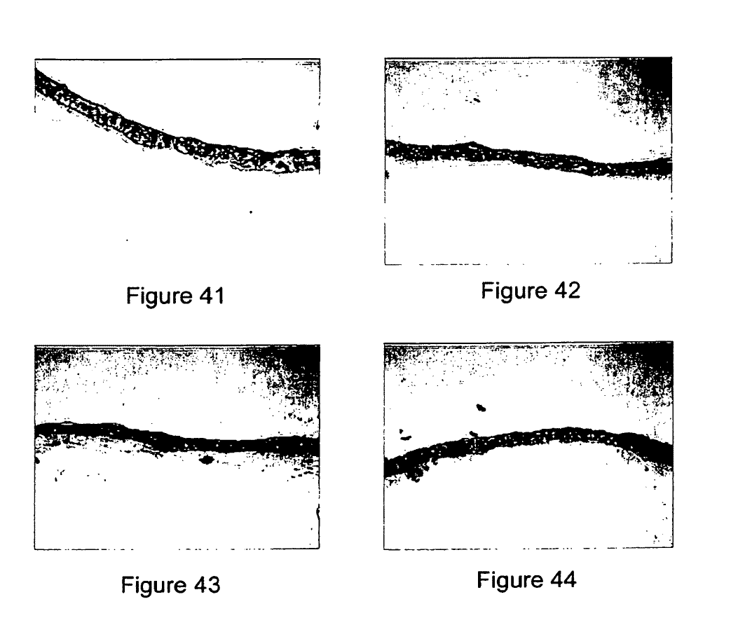

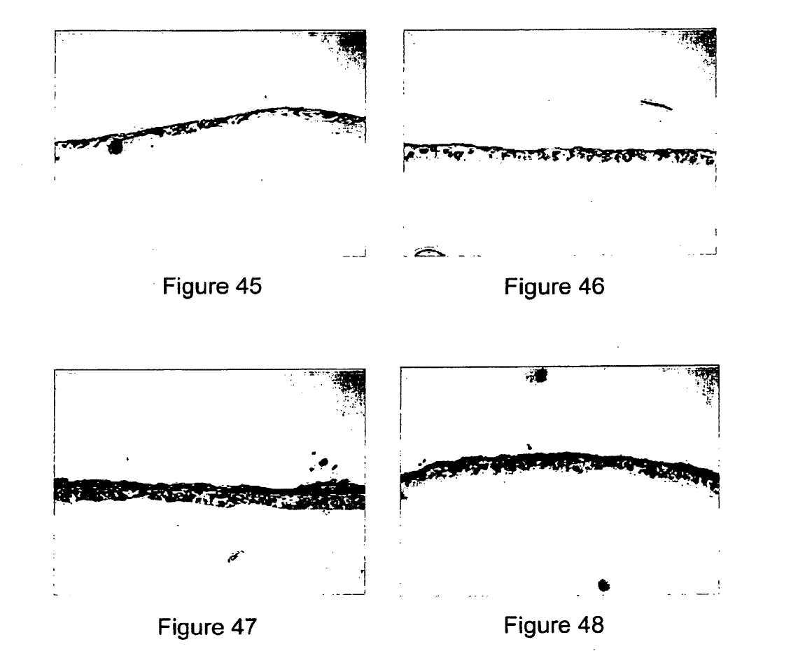

Images of immunostained sections following serum free storage of cultured HLEC at ambient temperature are shown in FIGS. 22 to 48. The pH and cell layers of tissue after 2 and 4 days storage are summarised in Table 9.

TABLE 9

| | Storage | | |
|---|---|---|---|
| | time | pH (before- | Cell layers, cell yield, |
| Medium | (days) | after) | oedema/detachment |
| Control (no | — | — | 3, many cells |
| storage) | | | |
| Optisol-GS | 2 | −7.73 | 3, many cells |
| MEM-Hepes | 2 | 7.38-7.36 | 4 (max 6), many cells |
| EpiLife | 2 | 7.68-7.81 | 2, few cells |
| Cnt20 | 2 | 7.28-7.60 | 2, few cells, oedema |
| PAA-Quantum | 2 | 7.69-8.41 | 2 (max 4), many cells |
| Optisol-GS | 4 | −7.84 | 3, many cells |
| MEM-Hepes | 4 | 7.38-7.27 | Few cells, cell detachment |
| EpiLife | 4 | 7.68-7.99 | Few cells, cell detachment |
| Cnt20 | 4 | 7.28-7.74 | 2, few cells, oedema |
| PAA-Quantum | 4 | 7.69-8.56 | 3, many cells |

3-week HLEC cultures served as controls and showed a multilayered epithelium of approximately 3 cell layers. Following storage for 2 and 4 days in Optisol-GS and PAA-Quantum, the multilayered structure was maintained, and there was no enlargement of intercellular spaces, detachment of epithelial cells, or detachment of the epithelia from the AM. The morphology was preserved after 2-days storage in MEM-Hepes, but few cells and detachment of epithelial cells were noticed after 4-days storage. Considerable detachment of epithelial cells was evident after storage in EpiLife, whereas few cells and intercellular oedema was observed following storage in Cnt-20.

In general, HLEC cultures demonstrated similar immunoreactivity of limbal stem cell (deltaNp63alpha), progenitor (p63 and K19), and differentiation (K3) markers following storage in Optisol-GS and PAA-Quantum. Weak nucleolar expression of deltaNp63α was present in all layers of cultured epithelium. P63 showed strong nuclear positivity in the basal and suprabasal layer, whereas K19 demonstrated weak cytoplasmic staining in the respective layers. K3 protein was moderately expressed in the basal and superficial layer after storage in PAA-Quantum, but only weakly expressed in the basal layer following storage in Optisol-GS.

### Example 5

### Sterility Testing of Storage Media

The purpose of this example was to show the microbiological sterility of cultured HLEC following eye bank storage. Sterility was tested using the blood bottle method.

### Method

### Microbiological Analysis of HLEC Storage Media

Human limbal explant cultures were prepared as described in Example 4 except that cultures were incubated for 21 days. Sterility testing of the storage media was performed using the blood bottle method as previously reported by Gain et al.^{100, 101} with some minor modifications. After one week storage, medium from the glass containers (n=23) were systematically sampled under a class II safety bench. 10-15 ml were injected into a Bactec Plus Aerobic/F bottle (Becton Dickinson, Cockeysville, Md., USA) containing 25 ml of enriched soybean-casein digest broth, 16% (wt/vol) non-ionic adsorbent resin and 1% (wt/vol) cationic exchange resins. 10-15 ml were injected in a Bactec Lytic/10 Anaerobic/F bottle (Becton Dickinson) containing 40 ml of soybean-casein digest broth, 0.26% (wt/vol) of saponin. The bottles were rocked continuously at 37° C. for 7 days in a Bactec 9240 incubator (Becton Dickinson) that detects rise in carbon dioxide due to microorganism development.

### Results

None of the 46 blood culture bottles (Bactec Plus Aerobic/F bottles (n=23) and Bactec Lytic/10 Anaerobic/F bottles (n=23)) were contaminated, giving a contamination rate of 0%.

### Example 6

### Long-Term Storage of Cultured HLEC

The purpose of this example was to demonstrate long term storage of cultured HLEC. 3-week HLEC cultures were organ cultured at 23° C. Viability was analyzed by CAM/EH-1-assay and the phenotypes were assessed by immunohistochemistry.

### Methods

### Human Limbal Explant Cultures

Human limbal explant cultures were prepared as described in Example 4 except that cultures were incubated for 21 days.

### Eye Bank Storage of Cultured HLEC

The HLEC cultures were subject to eye bank storage as previously reported.¹⁰² The medium was made of Dulbecco's modified Eagle's medium containing 7.5% sodium bicarbonate (Sigma-Aldrich), 8% FBS (Sigma-Aldrich), 50 μg/mL gentamicin (Sigma-Aldrich),100 μg/mL vancomycin (Abbott Laboratories, Abbott Park, Ill., USA), and 2.5 μg/mL amphotericin B (Sigma-Aldrich). HLEC cultures on iAM were stored at 23° C. for two weeks (n=16) or three weeks (n=17) with media change once a week under a class II safety bench.

### Live-Dead Viability Assays of Cultured HLEC Following Eye Bank Storage

Viability staining was performed as previously described. The number of live cells (green fluorescence) and dead cells (red fluorescence) was counted per 5 fields at 25-fold magnification, and the percentage of live cells was calculated. HLEC cultures (n=2) served as positive control for live cells and HLEC cultures exposed to methanol for 1 hour were used as a positive control for dead cells.

### Histology and Immunostaining of Cultured HLEC on iAM Following 2- and 3-Week Eye Bank Storage

HLEC cultures after 2-week storage (n=10) and 3-week storage (n=11) were fixed in neutral buffered 4% formaldehyde and embedded in paraffin. Serial sections of 5 μm were routinely stained with haematoxylin and eosin (H&E). Immunohistochemistry was performed with a panel of antibodies for markers of human ocular surface epithelia (Table 10). To visualize the immunoreactions, a standard peroxidase technique (DAB detection kit) was used in a Ventana ES Immunohistochemistry Instrument (Tucson, Ariz.). Optimal antibody dilutions were determined by titration using the positive controls recommended by the manufacturers. Histological evaluation and semiquantitative immunohistochemical localization of the epithelial markers were carried out by two independent investigators using a microscope at a magnification of ×400.

### Statistical Analysis

Data are presented as mean±SD. The Mann-Whitney test was applied to assess the difference in cell viability rate. A significance level of 5% was chosen and all data were analysed using the SPSS software package version 14.0.

### Results

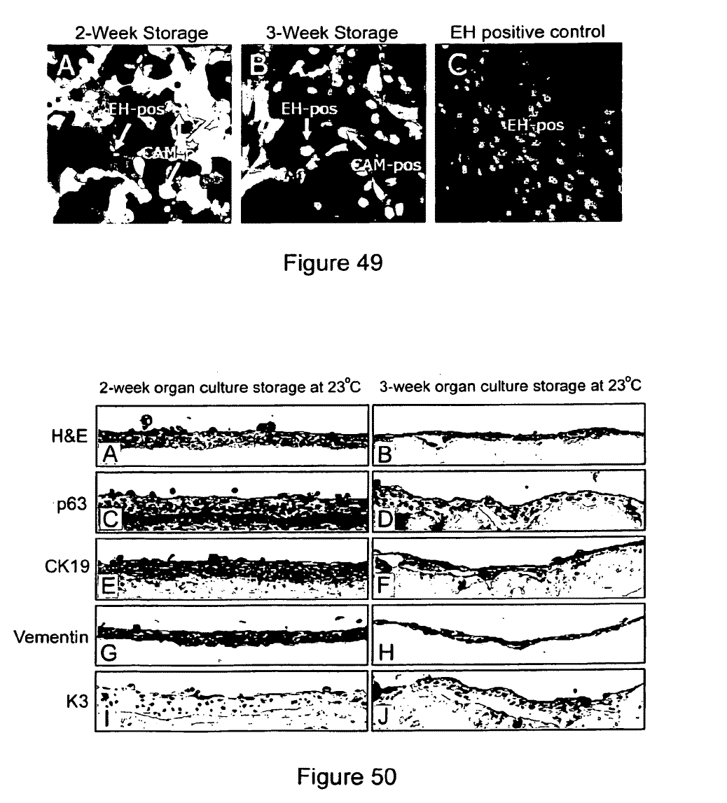

Basal layer viability of cultured limbal epithelial cells was 85.6%±13.5% after 2-week storage versus 52.7%±13.1% after 3-week storage (P<0.001, FIG. 49). FIG. 49 demonstrates viability staining of the basal layer of cultured HLEC after 2-week and 3-week storage.

The original multilayered structure was maintained in seven out of ten of the cultures after 2-week storage (FIG. 50A), but mostly lost in ten out of eleven cultures after three weeks with substantial detachment of superficial and suprabasal epithelial cells or detachment of the epithelia from the AM (FIG. 50B). Immunohistochemistry following 2- and 3-week storage revealed only minor changes and both intervals demonstrated a relatively undifferentiated phenotype with strong immunostaining of K19, Vimentin, and p63. CK3 was only slightly expressed in the (FIGS. 50C-50J, Table 10).

TABLE 10
Semiquantitative immunohistochemical localization of ocular surface markers in cultured
human limbal epithelial cells after 2-week and 3-week organ culture storage at 23° C.

| | | | | 2-week | | 3-week | |
|---|---|---|---|---|---|---|---|
| | | | | organ culture | | organ culture | |
| | | | Antibody | storage at 23° C.* | | storage at 23° C. [symbol] | |
| Antigen | Clone | Source | dilution | B | SB | B | SB |
| p63 | 4A4 | Dako* | 1:25 | +++ | + | ++ | ++ |
| K19 | RCK108 | Dako* | 1:200 | +++ | +++ | +++ | +++ |
| Vimentin | VIM 3B4 | Ventana Medical Systems† | RTU | +++ | +++ | +++ | +++ |
| Ki67 | MIB-1 | Dako* | 1:75 | 0 | 0/+ | 0/+ | 0/+ |
| K3 | AE5 | ImmuQuest‡ | 1:500 | 0 | 0/+ | 0 | 0/+ |
| K5 | XM26 | Novocastra Laboratories Ltd§ | 1:600 | +++ | +++ | +++ | +++ |
| K14 | LL02 | Novocastra Laboratories Ltd§ | 1:80 | +++ | +++ | +++ | +++ |
| Cx43 | Polyclonal | Sigma-Aldrich∥ | 1:500 | + | + | + | + |
| E-Cadherin | NCH-38 | Novocastra Laboratories Ltd§ | 1:25 | +++ | +++ | ++ | +++ |
| Integrin β1 | 7F10 | Novocastra Laboratories Ltd§ | 1:10 | +++ | ++ | + | + |

The immunoreactivity was graded as 0 (undetectable), + (weak positivity of >50% cells), ++ (intermediate positivity of >50% cells), +++ (strong positivity of >50% cells). All scores were assigned at a magnification of x400 by two independent experienced investigators blinded to the origin of the samples.
B: basal layer, SB: suprabasal layer; RTU: ready to use.
*Glostrup, Denmark
†Tucson, Arizona, USA
‡Cleveland, UK
§Newcastle, UK
∥St. Louis, MO [symbol]
indicates data missing or illegible when filed

### Example 7

### Depithelialization of the AM Prior to Storage

The purpose of this example was to demonstrate the effects of depithelialization of the amniotic membrane of cultured HLEC subject to eye bank storage. 3-week HLEC cultures on intact (iAM) or denuded amniotic membranes (dAM) were organ cultured at 23° C. Transmission and scanning electron microscopy were performed on iAM and dAM cultures following 1-week storage.

### Methods

### Human Limbal Explant Cultures

Human limbal explant cultures were prepared as described in Example 4 except that cultures were incubated for 21 days. Human AM were preserved in accordance with a method previously reported by Lee & Tseng¹ and according to the Declaration of Helsinki. Immediately prior to use, the AM was thawed, washed three times with sterile phosphate buffer solution (Sigma-Aldrich). Eight of the membranes were deprived of the amniotic epithelial cells by incubation with 0.02% ethylene diamine tetraacetic acid (Sigma-Aldrich) at 37° C. for 2 hours to loosen cellular adhesion, followed by gentle scraping using a cell scraper (Nalge Nunc International, Naperville, Ill., USA) as reported by Koizumi et al.³² The membranes were fastened as previously described.

### Eye Bank Storage of Cultured HLEC

The HLEC cultures were subject to eye bank storage as previously reported.¹⁰² The medium was made of Dulbecco's modified Eagle's medium containing 7.5% sodium bicarbonate (Sigma-Aldrich), 8% FBS (Sigma-Aldrich), 50 μg/mL gentamicin (Sigma-Aldrich),100 μg/mL vancomycin (Abbott Laboratories, Abbott Park, Ill., USA), and 2.5 μg/mL amphotericin B (Sigma-Aldrich). HLEC cultures on iAM (n=6) and dAM (n=8) were stored for one week.

### Quantitative Transmission Electron Microscopy Analysis on HLEC Cultures on iAM and dAM Following 1-Week Storage

Samples from HLEC cultures on iAM (n=6) and dAM (n=8) following 1-week storage were fixed in 2% glutaraldehyde in 0.2 M cacodylate buffer adjusted to pH 7.4, postfixed in 1% osmium tetroxide and dehydrated through a graded series of ethanol up to 100%. The tissue blocks were immersed in propylene oxide twice for 20 minutes and embedded in Epon. Ultrathin sections were cut on a Leica Ultracut Ultramicrotome UCT (Leica, Wetzlar, Germany) and examined using a Philips CM120 transmission electron microscope (Philips, Amsterdam, the Netherlands). A comparison of desmosome and hemidesmosome numbers was performed as previously described with some modifications. The number of desmosomes between neighbouring cells in the whole thickness of the epithelial layer was counted manually over a length of 120 μm in randomly selected regions. The number of hemidesmosomes at the basement membrane was quantified over randomly selected 20 μm distances. The counting of cellular attachments was performed by two independent investigators.

### Scanning Electron Microscopy of HLEC Cultures on iAM and dAM Following 1-Week Storage

Glutaraldehyde-fixed samples from HLEC cultures on iAM (n=6) and dAM (n=8) following 1-week storage were dehydrated in increasing ethanol concentrations and dried according to the critical point method (Polaron E3100 Critical Point Drier, Polaron Equipment Ltd., Watford, UK) using carbon dioxide as the transitional fluid. The specimens were fixed to carbon stubs and coated with a 300 Å thick layer of platinum in a Polaron E5100 sputter coater before being examined and photographed in a Philips XL30 ESEM electron microscope (Amsterdam, Netherlands).

### Statistical Analysis

Data are presented as mean±SD. The Mann-Whitney test was applied to assess the mean differences in cellular attachments. A significance level of 5% was chosen and all data were analysed using the SPSS software package version 14.0.

### Results

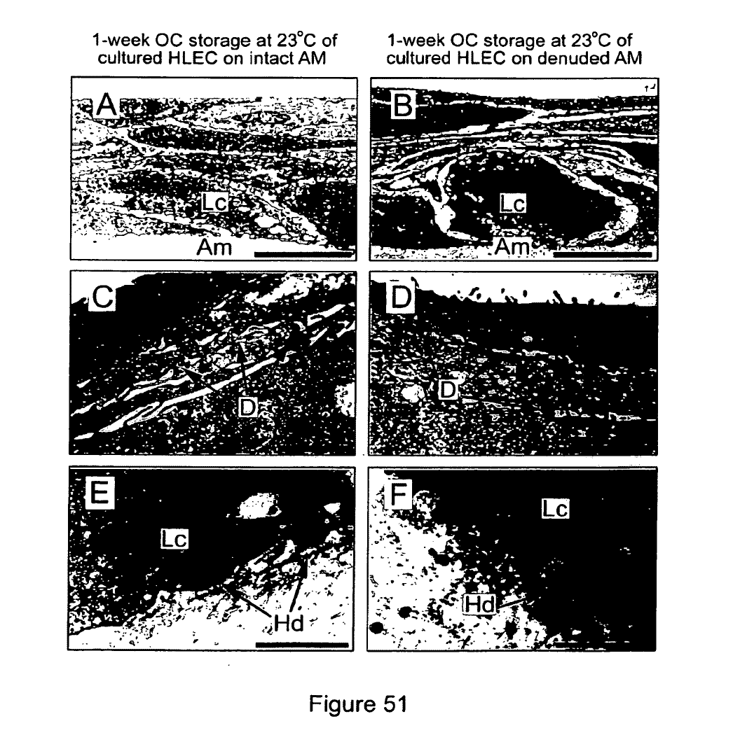

Following 1-week storage, both experimental groups demonstrated a multilayered epithelium. Irrespective of the presence of the amniotic epithelium (FIGS. 51A, 51B), the intercellular spaces were slightly increased, and numerous desmosomes (FIGS. 51C, 51D) and hemidesmosomes (FIGS. 51E, 51F) were observed. The total number of desmosomes per μm was 1.39±0.77 in HLEC cultures on iAM after 1-week eye-banking versus 0.98±0.45 in HLEC expanded on dAM after 1-week eye-banking (p=0.76). The total number of hemidesmosomes per μm in cultures on intact and denuded amniotic membrane was 0.87±0.34 and 0.78±0.31, p=0.70, respectively.

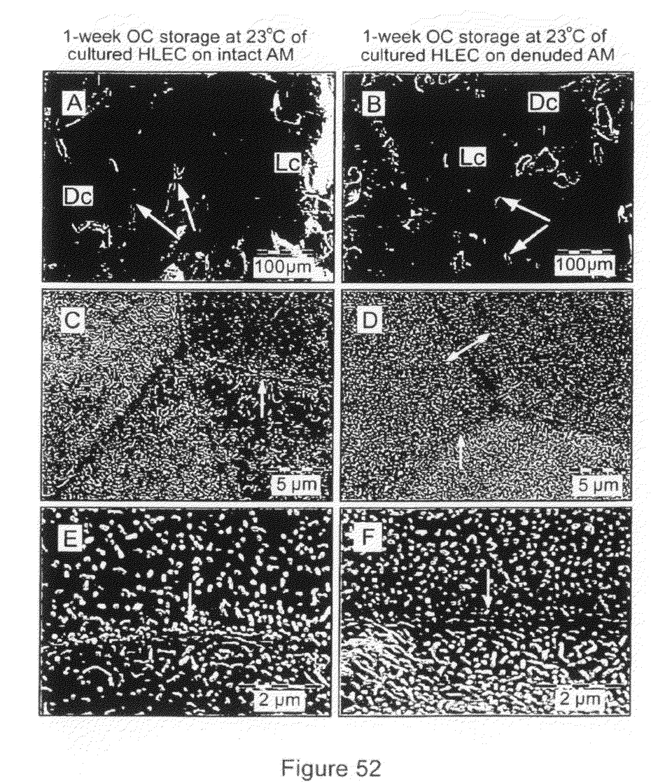

A confluent layer of light (Lc) and dark epithelial cells (Dc) with signs of cell separation (arrows) was observed in both groups (FIGS. 52A, 52B). Most of the epithelial cells were closely attached to each other with tightly opposed cell junctions and distinct cell borders (FIGS. 52C-E, arrows). However, cell separation (FIG. 52D, double arrow) and obscure cell borders (FIG. 52F, arrow) as demonstrated in cultured HLEC on denuded AM (FIG. 52D, F), occurred in both groups.

### Example 8

### Effect of Limbal Regional Origin

The purpose of this example was to demonstrate the differences between human limbal epithelial cells (HLEC) expanded from limbal explants of different origin along the corneal circumference.

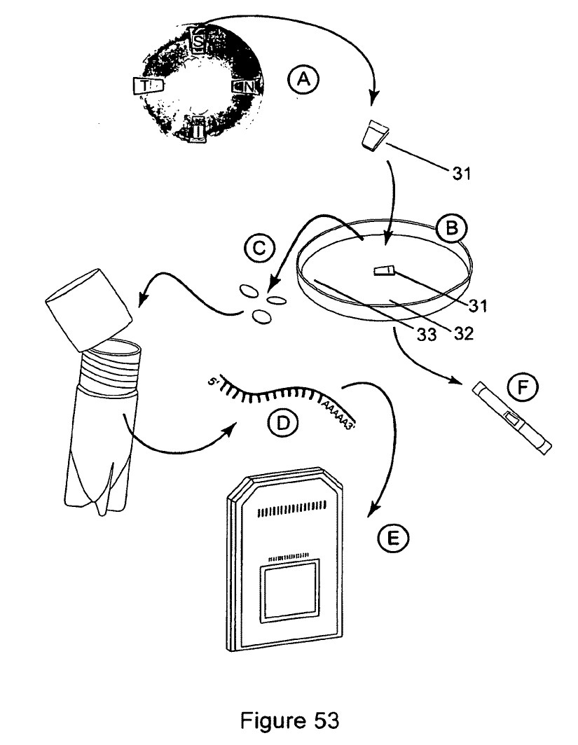

Referring to FIG. 53, a summary of the experimental design will now be provided. Corneoscleral explants 31 of 1-clock-hour width (i.e. 30°) were excised from the superior, nasal, inferior, and temporal limbal regions (A). HLEC were cultured for three weeks on intact amniotic membranes 32, fastened to polyester membrane 33, in supplemented hormonal epithelial medium (B). Discs of cultured epithelium were trephinated using a 5 mm biopsy punch and stored in cryo tubes at −80° C. until further use (C). RNA was extracted using QIAGEN RNeasy Micro Kit (D). 100 ng of total RNA was subjected to the GeneChip HT One-Cycle cDNA Synthesis Kit, and labelled and fragmented single stranded DNAs were hybridized to the GeneChip Human Gene 1.0 ST Array (E) for washing and staining. The remaining HLEC cultures were fixed in neutral buffered 4% formaldehyde and a rectangular specimen including the cultured epithelium and the explant was processed and embedded in paraffin (F).

### Materials and Methods

HLEC were cultured on amniotic membranes for 21 days from limbal explants from the superior, nasal, inferior, and temporal regions. The epithelia were characterised by light microscopy, whole genome transcript profiling using Affymetrix GeneChip Human 1.0 ST Array (Santa Clara, Calif., USA), and immunohistochemistry.

Dulbecco's modified Eagle's medium (DMEM), HEPES-buffered DMEM containing sodium bicarbonate and Ham's F12 (1:1), Hanks' balanced salt solution, fetal bovine serum (FBS), insulin-transferrin-sodium selenite media supplement, human epidermal growth factor, dimethyl sulfoxide, hydrocortisone, gentamycin, amphotericin B, beta-mercaptoethanol, and mouse anti-ABCG2 antibody (clone bxp21) were purchased from Sigma-Aldrich (St. Louis, Mo., USA). Dispase II was obtained from Roche Diagnostics (Basel, Switzerland), cholera toxin A subunit from Biomol (Exeter, UK), 5 mm biopsy punches from Kai Industries (Gifu, Japan), 6-0 C-2 monofilament sutures (Ethicon Ethilon) from Johnson & Johnson (New Brunswick, N.J.), 24 mm culture plate inserts (Netwell, 74 μm mesh size polyester membrane) from Costar Corning (New York, N.Y., USA), and vancomycin from Abbott Laboratories (Abbott Park, Ill., USA). Mouse anti-p63 antibody (clone 4A4), mouse anti-K19 antibody (clone RCK108), and anti-PCNA antibody (clone PC10) were obtained from Dako (Glostrup, Denmark), rabbit polyclonal anti-deltaNp63α antibody from Primm (Milano, Italy), mouse anti-vimentin antibody (clone VIM 3B4, ready to use) from Ventana Medical Systems (Tucson, Ariz., USA), mouse anti-Ki67 antibody (clone SP6) from LabVision Corporation (Fremont, Calif., USA), mouse anti-Nestin antibody (clone 10C2) from Santa Cruz Biotechnology, Santa Cruz, Calif., USA), and mouse anti-K3 antibody (clone AE5) from ImmuQuest (Cleveland, UK). The following antibodies were sourced from Novocastra Laboratories Ltd (Newcastle, UK): mouse anti-K5 (clone XM26), mouse anti-E-cadherin (clone NCH-38), and mouse anti-integrin f31 (clone 7F10). EnVision Peroxidase detection system was purchased from Dako, cryotubes from Nunc (Roskilde, Denmark), QIAGEN RNeasy Micro Kit and RLT buffer from QIAGEN (Hilden, Germany), and 1.5 mL microcentrifuge tubes from Eppendorf (Hamburg, Germany). GeneChip HT One-Cycle cDNA Synthesis Kit, GeneChip HT IVT Labeling Kit, and GeneChip Human Gene 1.0 ST Arrays were from Affymetrix (Santa Clara, Calif., USA).

### Human Tissue Preparation

Human tissue was handled according to the Declaration of Helsinki. Oriented cadaveric human corneas with research consent were obtained from the Centro de Oftalmologia Barraquer (Barcelona, Spain). The study was conducted on eight cadaveric human corneas obtained from four donors (mean age, 74.8 years (range, 56-83); mean time from death to enucleation, 8.6 hours (range, 6-11), time from death to culture, 7 days (range, 3.5-11.5)). The limbal tissue was prepared as previously reported by Meller et al.² The tissue was rinsed three times with DMEM containing 50 μg/mL gentamicin and 1.25 μg/mL amphotericin B. After careful elimination of excessive sclera, conjunctiva, iris, and corneal endothelium, the remaining tissue was placed in a culture dish and exposed for 10 minutes to Dispase II in Mg²⁺ and Ca²⁺ free Hanks' balanced salt solution at 37° C. under humidified 5% carbon dioxide and thereafter carefully rinsed with DMEM containing 10% fetal bovine serum. From the superior, nasal, inferior, and temporal meridians, a corneoscleral explant of 1-clock-hour width was excised using a steel blade (FIG. 53).

### Human Limbal Explant Cultures on Intact Amniotic Membranes

Human amniotic membranes (AM) were preserved in accordance with a method previously reported by Lee & Tseng¹ and according to the Declaration of Helsinki. After thawing at room temperature, devitalised intact AM with the epithelial side facing up was fastened to the polyester membrane of a culture plate insert using monofilament sutures as previously reported.^{46,96} On the center of each AM insert, the explants were cultured in supplemented hormonal epithelial medium with the epithelial side facing down.⁹⁷ The medium was made of HEPES-buffered DMEM containing sodium bicarbonate and Ham's F12 (1:1) and was supplemented with 5% FBS, 0.5% dimethyl sulfoxide, 2 ng/m human epidermal growth factor, 5 μg/mL insulin, 5 μg/mL transferrin, 5 ng/mL selenium, 3 ng/mL hydrocortisone, 30 ng/mL cholera toxin, 50 μg/mL gentamycin, and 1.25 μg/mL amphotericin B. Cultures were incubated for 3 weeks at 37° C. in an atmosphere of humidified 5% carbon dioxide and 95% air, and the medium was changed every 2 to 3 days.

### RNA Isolation

The culture plate inserts were transferred from the culture plates to modelling wax plates, and disks of cultured epithelium adherent to the underlying polyester membrane were trephinated from cultures of superior (n=8), nasal (n=8), inferior (n=8), and temporal (n=8) origin using a 5 mm biopsy punch and stored in cryo tubes at −80° C. until further use (FIG. 53). Samples (n=2) of amniotic membranes were processed by the similar procedure to assess the RNA level in devitalised amniotic epithelium without cultured HLEC. The tissue disks were thawed and total RNA was extracted with QIAGEN RNeasy Micro Kit according to the manufacturer's protocol. 350 μl of RTL buffer with beta-mercaptoethanol was added to the disks in microcentrifuge tubes and subjected to vortexing for 2 minutes. No QIAshredder Spin Column or equivalents were used. RNA concentration and purity was determined through measurement of A260/A280 ratios with the Nano prop ND-1000 Spectrophotometer (Thermo Fisher Scientific, Wilmington, Del., USA). Confirmation of RNA quality was assessed by use of the Agilent 2100 Bioanalyzer and RNA 6000 Nano Assay (Agilent Technologies, Santa Clara, Calif., USA). All the RNA samples had high quality and showed no signs of DNA contamination or RNA degradation. RNA samples were immediately frozen and stored at −80° C.

### Chip Hybridization

100 ng of total RNA was subjected to the GeneChip HT One-Cycle cDNA Synthesis Kit and GeneChip HT IVT Labeling Kit following the manufacturer's recommended protocol for whole genome gene expression analysis (Affymetrix). Labelled and fragmented single stranded cRNA were hybridized to the GeneChip Human Gene 1.0 ST Arrays (28869 genes). The arrays were washed and stained using FS-450 fluidics station (Affymetrix). The signal intensities were detected by Hewlett Packard Gene Array Scanner 3000 7G (Hewlett Packard, Palo Alto, Calif., USA).

### Signal Normalization

The scanned images were processed using GCOS 1.4 (Affymetrix). The CEL files were imported into ArrayAssist Advanced Software ver.5.5.1 (lobion Informatics, La Jolla, Calif., USA) and normalized using the Exon IterPLIER algorithm to calculate relative signal values for each probe set. In addition, quantile normalization was performed and a variance stabilization factor of 16 was used.

### Histology and Immunostaining

Following trephination of epithelial disks for gene analysis, the remaining tissue was fixed in neutral buffered 4% formaldehyde (FIG. 53). A rectangular sample including the limbal explant and cultured epithelium was trimmed and embedded in paraffin. Serial sections of 5 μm from samples of superior (n=8), nasal (n=8), inferior (n=8), and temporal (n=8) origin were routinely stained with haematoxylin and eosin (H&E). As previously reported, the number of cell layers in cultured epithelia was counted by two independent investigators at regular intervals (50 μm) from the limbal explant margin to the leading edge of the epithelial outgrowth, using iTEM software (Soft Imaging System; Olympus, Miinster, Germany).⁹⁷ Serial 5 μm sections were immunostained with antibodies recognizing p63 (1:25 dilution), deltaNp63α antibody (1:200), ABCG2 (1:80), K19 (1:200), Vimentin (ready to use), Integrin β1 (1:10), Ki67 (1:75), PCNA (1:1500), Nestin (1:80), K3 (1:500), K5 (1:600), and E-Cadherin (1:25) in samples of superior (n=4), nasal (n=4), inferior (n=4), nd temporal (n=4) origin (Table 11). Detection was performed with EnVision Peroxidase with an automated immunostaining system (LabVision 360 Autostainer, LabVision Corporation). Optimal antibody dilutions were determined by titration using the positive controls recommended by the manufacturers.

### Statistical Analysis

The mean differences in cell layers and RNA yield between the experimental groups were tested by using the Mann-Whitney test (SPSS ver.14.0; SPSS Inc., Chicago, Ill., USA). P<0.05 was considered significant. For gene expression comparisons of different regions, class comparison analysis was performed using univariate F-test and a nominal significance level of 0.001 (BRB-ArrayTools ver.3.6.0, National Cancer Institute, National Institutes of Health, USA). Genes were excluded when less than 20% of expression data had at least a 1.5-fold change in either direction from gene's median value.

### Results

### Histology

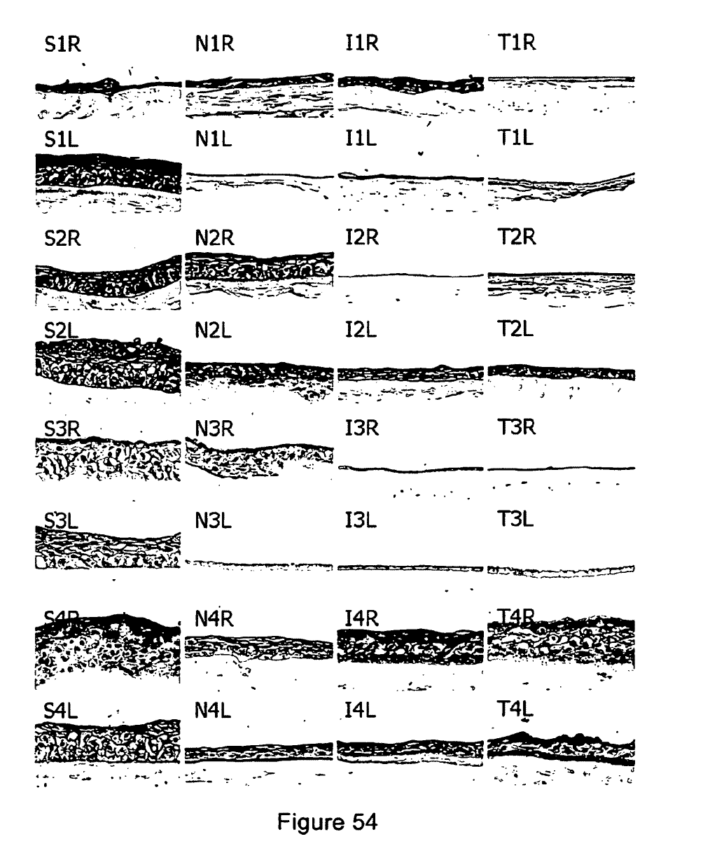

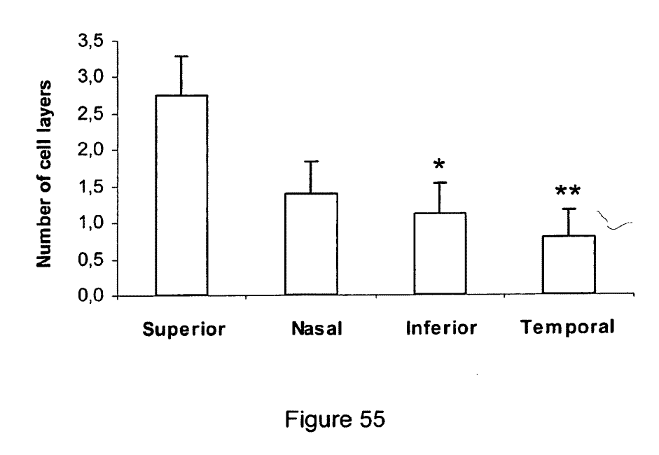

7/8 cultures of superior origin generated a stratified multilayered epithelium (>2 cell layers) versus 6/8 cultures of nasal origin, 4/8 cultures of inferior origin, and 3/8 cultures of temporal origin (FIG. 54). Most of the epithelia in the superior group demonstrated an epithelium consisting of basal column-shaped cells, suprabasal cuboid wing cells, and flat squamous superficial cells. The number of cell layers was significantly higher in cultures of superior origin compared with cultures of inferior origin (P=0.02) and temporal origin (P=0.01) (FIG. 55, Table 11). The differences in cell layers between the nasal, inferior, and temporal groups were not significant.

### RNA Isolation

The mean RNA yield was highest in cultures of superior origin and lowest in cultures derived from the temporal region (Table 12). Extracts representing all donors from 7 cultures of superior and nasal origin, 6 cultures of inferior origin, and 3 cultures of temporal origin provided sufficient RNA for microarray analysis. The RNA yield extracted from devitalised intact amniotic epithelium was below the acceptable range of input amount thus eliminating amniotic epithelial RNA as a source of error in the interpretation of the microarray data.

### Identifying Differentially Expressed Genes in HLEC Cultures of Different Donors and Limbal Origin

Few genes (<1‰ of 28869 tested) were differentially and significantly expressed among the four regions. Of the 1989 genes that passed filtering criteria when performing class comparison analysis, three genes, Tripartite Motif Containing 36 (TRIM36), Odd Skipped Related 2 (DROSOPHILA) (OSR2), and Ras Homolog Gene Familiy, Member U (RHOU) were significantly and differentially expressed among the four regions (Table 14). The expression of proposed limbal stem cell, progenitor, proliferation, and differentiation marker genes was not significantly changed when comparing HLEC cultures of different limbal origin (Table 13).

### Immunophenotypical Analysis

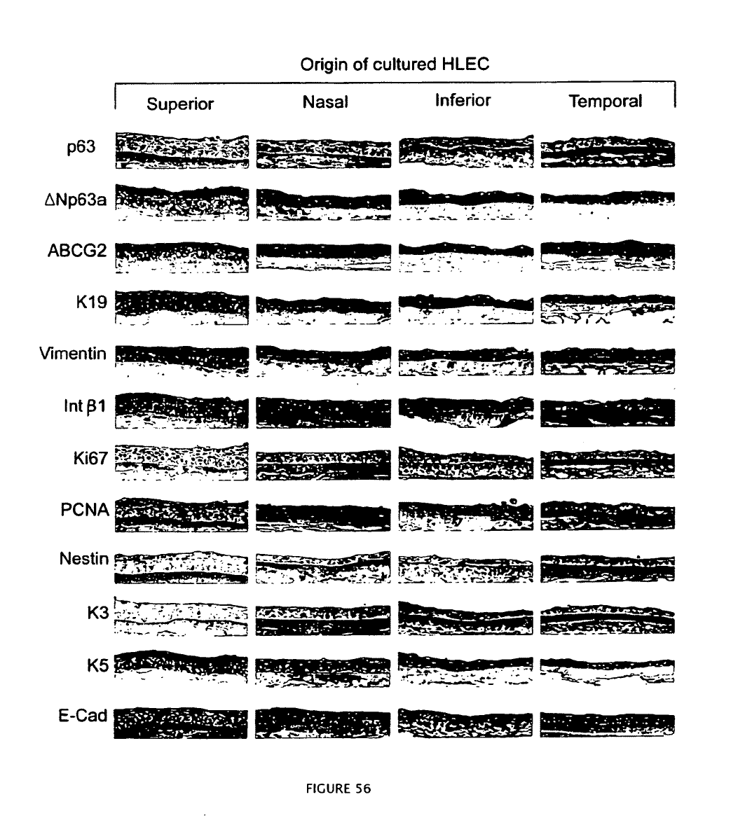

HLEC cultures of different limbal origin demonstrated similar immunoreactivity of proposed limbal stem cell and progenitor markers (FIG. 56). Weak nucleolar expression of deltaNp63α and membranous expression of ABCG2 was present in all layers of cultured epithelium. P63, K19 and vimentin showed strong nuclear/cytoplasmic positivity in the basal and suprabasal layer, whereas integrin β1 was weakly expressed in cell membranes of basal cells. The nuclear proliferation markers Ki67 and PCNA and differentiation markers were equally expressed at the protein level in HLEC cultures regardless of limbal origin. Intermediate cytoplasmic K5 expression was noticed in all cell layers, and E-Cadherin showed intermediate membranous immunostaining predominantly in the suprabasal and superficial layers. K3 protein and nestin were absent in all layers of cultured epithelium.

TABLE 11
Cell layers in cultured human limbal epithelial cells of
superior, nasal, inferior, and temporal limbal origin

| | | | | | Percentage of | |
|---|---|---|---|---|---|---|
| | | | Std. | | samples with | |
| Origin | n | Mean | Deviation | Maximum | A1 = 0 | P* |
| Superior | 8 | 2.8 | 1.5 | 4.9 | | — |
| Nasal | 8 | 1.4 | 1.3 | 3.0 | | 0.07 |
| Inferior | 8 | 1.1 | 1.2 | 2.5 | | 0.02 |
| Temporal | 8 | 0.8 | 1.1 | 2.3 | | 0.01 |

The number of cell layers in cultured epithelia was counted by two independent investigators at regular intervals (50 μm) from the limbal explant margin to the leading edge of the epithelial outgrowth, using iTEM software (Soft Imaging System; Olympus, Münster, Germany).
*P-values were calculated by testing the number of cell layers of the individual experimental group against the number of cell layers in HLEC cultures of superior origin using the Mann-Whitney test.

TABLE 12
RNA concentration (ng/μL) in cultured human limbal epithelial
cells of superior, nasal, inferior, and temporal limbal origin

| Origin | n | Mean | SD | Minimum | Maximum | P* |
|---|---|---|---|---|---|---|
| Superior | 8 | 209 | 149 | 0 | 468 | — |
| Nasal | 8 | 136 | 148 | 0 | 434 | 0.40 |
| Inferior | 8 | 103 | 78 | 0 | 207 | 0.20 |
| Temporal | 8 | 85 | 115 | 0 | 316 | 0.11 |

Total RNA from 5 mm trephinated disks of cultured epithelium was extracted using QIAGEN RNeasy Micro Kit according to manufacturers' instructions. RNA concentration and purity was determined through measurement of A260/A280 ratios with the Nano prop ND-1000 Spectrophotometer (Thermo Fisher Scientific, Wilmington, Del., USA). Confirmation of RNA quality was assessed by use of the Agilent 2100 Bioanalyzer and RNA 6000 Nano Assay (Agilent Technologies, Santa Clara, Calif., USA). * Calculated by testing the RNA concentration of the individual group against the RNA yield in HLEC cultures of superior origin using the Mann-Whitney test.

TABLE 13
Limbal stem cell, progenitor, proliferation, and differentiation
marker genes that were equally expressed in cultured HLECs
irrespective of limbal explant origin (Fold change < 2)

| | | Affymetrix |
|---|---|---|
| Marker | Gene symbol | probe set ID |
| A-enolase* | ENO1 | 7912198 |
| Keratin K3* | KRT3 | 7963523 |
| Keratin K5* | KRT5 | 7963427 |
| Keratin K12* | KRT12 | 8015115 |
| Keratin K14* | KRT14 | 8015366 |
| Keratin K15† | KRT15 | 8015337 |
| Keratin K19* | KRT19 | 8015349 |
| Vimentin* | VIM | 7926368 |
| Nestin* | NES | 7921088 |
| p63* | TP73L | 8084766 |
| PCNA‡ | PCNA | 8064844 |
| Ki67‡ | MKI67 | 7937020 |
| ABCG2* | ABCG2 | 8101675 |
| Connexin 43* | GJA1 | 8113467 |
| E-cadherin* | CDH1 | 7996837 |
| P-cadherin† | CDH3 | 7996819 |
| EGF-R* | EGFR | 8132860 |
| HGF-R* | MET | 8135601 |
| Integrin α2* | ITGA2 | 8105267 |
| Integrin α3* | ITGA3 | 8008237 |
| Integrin α6* | ITGA6 | 8046380 |
| Integrin α9* | ITGA9 | 8078619 |
| Integrin αv* | ITGAV | 8046861 |
| Integrin β1* | ITGB1 | 7932966 |
| Integrin β2* | ITGB2 | 8070826 |
| Integrin β4* | ITGB4 | 8009951 |
| Integrin β5* | ITGB5 | 8090162 |
| KGF-R bek* | FGFR2 | 7936734 |
| TrkA* | NTRK1 | 7906244 |
| TGF-β-RI* | TGFB1 | 8037005 |
| TGF-β-RII* | TGFB2 | 7909789 |
| Transferrin-R | TFRC | 8093053 |
| CD71* | | |
| Wnt-4† | WNT4 | 7913547 |

*Schlotzer-Schrehardt and Kruse⁵⁴
†Figueira et al.⁵⁵
‡General proliferation markers

TABLE 14
Class Comparison Analysis with Normalized Log-Transformed Gene Expressions
for Significant Genes (p < 0.05) in cultured HLEC of different limbal origin
Class
Superior origin

| | | Donor 1 | | | | | | |
|---|---|---|---|---|---|---|---|---|
| Gene symbol | Unique ID | Right eye | D1L | D2R | D2L | D3R | D4R | D4L |
| TRIM36 | 8113577 | 6.415 | 6.931 | 7.031 | 6.642 | 6.065 | 6.757 | 7.087 |
| OSR2 | 8147573 | 6.147 | 5.87 | 6.915 | 7.422 | 6.179 | 5.857 | 5.749 |
| RHOU | 7910387 | 6.425 | 5.606 | 6.639 | 6.457 | 6.627 | 6.896 | 6.976 |

Class
Nasal origin

| Gene symbol | Unique ID | D1R | D1L | D2R | D2L | D4R | D4L |
|---|---|---|---|---|---|---|---|
| TRIM36 | 8113577 | 5.95 | 5.863 | 5.886 | 5.349 | 5.882 | 5.448 |
| OSR2 | 8147573 | 7.346 | 7.238 | 7.913 | 7.157 | 8.05 | 7.343 |
| RHOU | 7910387 | 5.082 | 5.715 | 5.153 | 5.245 | 6.212 | 5.096 |

Class
Inferior origin

| Gene symbol | Unique ID | D1R | D1L | D2L | D3R | D4R | D4L |
|---|---|---|---|---|---|---|---|
| TRIM36 | 8113577 | 5.87 | 5.934 | 5.936 | 6.025 | 5.867 | 5.236 |
| OSR2 | 8147573 | 7.108 | 7.553 | 6.826 | 6.191 | 6.58 | 6.691 |
| RHOU | 7910387 | 5.314 | 5.634 | 5.47 | 6.402 | 5.892 | 5.544 |

Class
Temporal origin

| Gene symbol | Unique ID | D2L | D4R | D4L |
|---|---|---|---|---|
| TRIM36 | 8113577 | 6.024 | 5.163 | 5.054 |
| OSR2 | 8147573 | 7.392 | 7.917 | 7.964 |
| RHOU | 7910387 | 5.764 | 5.09 | 5.369 |

Number of genes that passed filtering criteria: 1989
Type of univariate test used: F-test (with random variance model)
Nominal significance level of each univariate test: 0.001

### Discussion

This example compared histology, whole genome profiles, and phenotype of cultured HLEC originating from different regions along the limbal circumference. Morphologically, HLEC cultures of superior origin yielded a significantly higher number of cell layers compared to cultures of inferior and temporal origin. No evidence of major transcriptional or phenotypical differences was found in cultured HLEC of different limbal origin.

In the example, histological analysis demonstrated that ex vivo cultured HLEC may be generated from limbal explants of superior, nasal, inferior, and temporal origin, which implicates the presence of cells with a proliferative potential in the respective limbal regions. However, the HLEC cultures differed significantly with regard to epithelial stratification in favour of explants from the superior region. Moreover, the success rate in terms of formation of a confluent stratified epithelium and RNA yield tended to be higher in HLEC cultures of superior origin. The observed differences indicate that limbal explants of superior origin have a higher proliferative potential in culture compared with explants of other limbal origin. The findings are consistent with the study by Wiley et al. suggesting that the superior region has a higher pool of epithelial cells with stem cell-like characteristics, and hence a higher proliferative potential.¹⁰³ The mitotic activity is, however, reported to be the same in superior, inferior, lateral, and medial limbus.^{104, 105, 112, 113}

It has been suggested that a multilayered cultured corneal epithelium may indicate a high grade of differentiation.^{31,32} However, the negative expression of the differentiation markers K3 and nestin in cultured HLEC irrespective of limbal origin in the example, does not support the supposition that cultured HLEC of superior origin should be more differentiated. On the other hand, a multilayered epithelium may be superior to a single layered epithelium with regard to the clinical outcome following transplantation. ^{31,35} A multilayered corneal epithelial graft with intercellular desmosomes providing high mechanical strength should better resist the mechanical stress and abrasions associated with the transplantation. Furthermore, a high content of undifferentiated cells should improve the regeneration of the corneal surface of the recipient.

In the example, less than 1‰ of the 28869 genes tested showed changes over 2-fold indicating a strong homogeneity in gene expression when comparing HLEC cultures of the respective limbal origin. The gene expression level of previously reported limbal stem cell, progenitor, and differentiation markers^{98,99} and proliferation markers was not significantly changed. The results from the phenotypical analysis were in agreement with the gene expression analysis. Limbal stem cell, progenitor, proliferation, and differentiation protein markers were homogenously expressed regardless of limbal origin, collectively demonstrating an undifferentiated phenotype.

TRIM36, OSR2, and RHOU were differentially and significantly expressed among the four regions. Short et al found TRIM36 to be associated with the microtubule cytoskeleton, however, the exact function of TRIM 36 is not yet clear.¹⁰⁶ OSR2 plays a key role in osteoblastic cell proliferation¹⁰⁷. Moreover, OSR2 play a part in palate growth and morphogenesis as well as kidney development.^{108,109}. RHOU belongs to the group of Rho GTPases, which are known to play central roles in the control of cell adhesion and migration, cell cycle progression, growth, and differentiation.¹¹⁰. Ory et al found that migration distances were increased in cells expressing activated RhoU and decreased when RhoU was knocked-down. ¹¹¹ Hence, all the three genes are collectively involved in morphogenesis.

Taking these data together, the superior region is the preferred site for limbal harvesting for both live and dead donors.

### REFERENCES

1. Lee S H, Tseng S C. Amniotic membrane transplantation for persistent epithelial defects with ulceration. Am J Ophthalmol 1997; 123:303-12.
2. Meller D, Pires R T, Tseng S C. Ex vivo preservation and expansion of human limbal epithelial stem cells on amniotic membrane cultures. Br J Ophthalmol 2002; 86:463-71.
3. Hershey F B, Cruickshank C N, Mullins L I. The quantitative reduction of 2,3,5-triphenyl tetrazolium chloride by skin in vitro. J Histochem Cytochem 1958; 6:191-6.
4. Bravo D, Rigley T H, Gibran N, et al. Effect of storage and preservation methods on viability in transplantable human skin allografts. Burns 2000; 26:367-78.
5. Kito K, Kagami H, Kobayashi C, et al. Effects of cryopreservation on histology and viability of cultured corneal epithelial cell sheets in rabbit. Cornea 2005; 24:735-41.
6. James S E, Rowe A, Ilari L, et al. The potential for eye bank limbal rings to generate cultured corneal epithelial allografts. Cornea 2001; 20:488-94.
7. Joseph A, Powell-Richards A O, Shanmuganathan V A, et al. Epithelial cell characteristics of cultured human limbal explants. Br J Ophthalmol 2004; 88:393-8.
8. Shanmuganathan V A, Rotchford A P, Tullo A B, et al. Epithelial proliferative potential of organ cultured corneoscleral rims; implications for allo-limbal transplantation and eye banking. Br J Ophthalmol 2006; 90:55-8.
9. Armitage W J, Easty D L. Factors influencing the suitability of organ-cultured corneas for transplantation. Invest Ophthalmol Vis Sci 1997; 38:16-24.
10. Summerlin W T, Miller G E, Harris J E, et al. The organ-cultured cornea: an in vitro study. Invest Ophthalmol 1973; 12:176-80.
11. Sachs U, Goldman K, Valenti J, et al. Corneal storage at room temperature. Arch Ophthalmol 1978; 96:1075-7.
12. Tamaki K, Vamell E D, Kaufman HE. K-Sol corneal preservation at room temperature. Br J Ophthalmol 1988; 72:370-6.
13. Reim M, Hesse R, Pietruschka G. The metabolism of organ cultures of cornea in TC 199 with added dextran 500 or hydroxyethyl starch 450. Klin Monatsbl Augenheilkd 1990; 196:76-80.
14. Sandboe F D, Medin W, Froslie K F. Influence of temperature on corneas stored in culture medium. A comparative study using functional and morphological methods. Acta Ophthalmol Scand 2003; 81:54-9.
15. Borderie V M, Kantelip B M, Delbosc B Y, et al. Morphology, histology, and ultrastructure of human C31 organ-cultured corneas. Cornea 1995; 14:300-10.
16. Van Horn D L, Doughman D J, Harris J E, et al. Ultrastructure of human organcultured cornea. II. Stroma and epithelium. Arch Ophthalmol 1975; 93:275-7.
17. Grueterich M, Espana E, Tseng S C. Connexin 43 expression and proliferation of human limbal epithelium on intact and denuded amniotic membrane. Invest Ophthalmol Vis Sci 2002; 43:63-71.
18. Hernandez Galindo E E, Theiss C, Steuhl K P, et al. Gap junctional communication in microinjected human limbal and peripheral corneal epithelial cells cultured on intact amniotic membrane. Exp Eye Res 2003; 76:303-14.
19. Ban Y, Cooper L J, Fullwood N J, et al. Comparison of ultrastructure, tight junctionrelated protein expression and barrier function of human corneal epithelial cells cultivated on amniotic membrane with and without air-lifting. Exp Eye Res 2003; 76:735-43.
20. Pellegrini G, Traverso C E, Franzi A T, Zingirian M, Cancedda R, De L M. Long-term restoration of damaged corneal surfaces with autologous cultivated corneal epithelium. Lancet. 1997; 349:990-993.
21. Tsai R J, Li L M, Chen J K. Reconstruction of damaged corneas by transplantation of autologous limbal epithelial cells. N Engl J of Med. 2000; 343:86-93.
22. Schwab I R, Reyes M, Isseroff R R. Successful transplantation of bioengineered tissue replacements in patients with ocular surface disease. Cornea. 2000; 19:421-426.
23. Koizumi N, Inatomi T, Suzuki T, Sotozono C, Kinoshita S. Cultivated corneal epithelial stem cell transplantation in ocular surface disorders. Ophthalmology. 2001; 108:1569-1574.
24. Shimazaki J, Aiba M, Goto E, Kato N, Shimmura S, Tsubota K. Transplantation of human limbal epithelium cultivated on amniotic membrane for the treatment of severe ocular surface disorders. Ophthalmology. 2002; 109:1285-1290.
25. Ti S E, Grueterich M, Espana E M, Touhami A, Anderson D F, Tseng S C. Correlation of long term phenotypic and clinical outcomes following limbal epithelial transplantation cultivated on amniotic membrane in rabbits. Br J Ophthalmol. 2004; 88:422-427.
26. Schwab I R. Cultured corneal epithelia for ocular surface disease. Trans Am Ophthalmol Soc. 1999; 97:891-986.
27. Koizumi N, Inatomi T, Quantock A J, Fullwood N J, Dota A, Kinoshita S. Amniotic membrane as a substrate for cultivating limbal corneal epithelial cells for autologous transplantation in rabbits. Cornea. 2000; 19:65-71.
28. Grueterich M, Tseng S C. Human limbal progenitor cells expanded on intact amniotic membrane ex vivo. Arch Ophthalmol. 2002; 120:783-790.
29. Meller D, Pires R T, Tseng S C. Ex vivo preservation and expansion of human limbal epithelial stem cells on amniotic membrane cultures. Br J Ophthalmol. 2002; 86:463-471.
30. Lindberg K, Brown M E, Chaves H V, Kenyon K R, Rheinwald J G. In vitro propagation of human ocular surface epithelial cells for transplantation. Invest Ophthalmol Vis Sci. 1993; 34:2672-2679.
31. Koizumi N, Cooper L J, Fullwood N J, et al. An evaluation of cultivated corneal limbal epithelial cells, using cell-suspension culture. Invest Ophthalmol Vis Sci. 2002; 43:2114-2121.
32. Koizumi N, Fullwood N J, Bairaktaris G, Inatomi T, Kinoshita S, Quantock A J. Cultivation of corneal epithelial cells on intact and denuded human amniotic membrane. Invest Ophthalmol Vis Sci. 2000; 41:2506-2513.
33. Grueterich M, Espana E, Tseng S C. Connexin 43 expression and proliferation of human limbal epithelium on intact and denuded amniotic membrane. Invest Ophthalmol Vis Sci. 2002; 43:63-71.
34. Koizumi N, Inatomi T, Suzuki T, Sotozono C, Kinoshita S. Cultivated corneal epithelial transplantation for ocular surface reconstruction in acute phase of Stevens-Johnson syndrome. Arch Ophthalmol. 2001; 119:298-300.
35. Koizumi N, Rigby H, Fullwood N J, et al. Comparison of intact and denuded amniotic membrane as a substrate for cell-suspension culture of human limbal epithelial cells. Graefes Arch Clin Exp Ophthalmol. 2007; 245:123-134.
36. Rama P, Bonini S, Lambiase A, et al. Autologous fibrin-cultured limbal stem cells permanently restore the corneal surface of patients with total limbal stem cell deficiency. Transplantation. 2001; 72:1478-1485.
37. Nishida K, Yamato M, Hayashida Y, et al. Functional bioengineered corneal epithelial sheet grafts from corneal stem cells expanded ex vivo on a temperature-responsive cell culture surface. Transplantation. 2004; 77:379-385.
38. Pino C J, Haselton F R, Chang M S. Seeding of corneal wounds by epithelial cell transfer from micropatterned PDMS contact lenses. Cell Transplant. 2005; 14:565-571.
39. Higa K, Shimmura S, Kato N, et al. Proliferation and differentiation of transplantable rabbit epithelial sheets engineered with or without an amniotic membrane carrier. Invest Ophthalmol Vis Sci. 2007; 48:597-604.
40. Di G N, Chui J, Wakefield D, Coroneo M T. Cultured human ocular surface epithelium on therapeutic contact lenses. Br J Ophthalmol. 2007; 91:459-464.
41. Ban Y, Cooper L J, Fullwood N J, et al. Comparison of ultrastructure, tight junction-related protein expression and barrier function of human corneal epithelial cells cultivated on amniotic membrane with and without air-lifting. Exp Eye Res. 2003; 76:735-743.
42. Nakamura T, Endo K, Cooper L J, et al. The successful culture and autologous transplantation of rabbit oral mucosal epithelial cells on amniotic membrane. Invest Ophthalmol Vis Sci. 2003; 44:106-116.
43. Nakamura T, Inatomi T, Sotozono C, Amemiya T, Kanamura N, Kinoshita S. Transplantation of cultivated autologous oral mucosal epithelial cells in patients with severe ocular surface disorders. Br J Ophthalmol. 2004; 88:1280-1284.
44. Hayashida Y, Nishida K, Yamato M, et al. Ocular surface reconstruction using autologous rabbit oral mucosal epithelial sheets fabricated ex vivo on a temperature-responsive culture surface. Invest Ophthalmol Vis Sci. 2005; 46:1632-1639.
45. Nakamura T, Ang L P, Rigby H, et al. The use of autologous serum in the development of corneal and oral epithelial equivalents in patients with Stevens-Johnson syndrome. Invest Ophthalmol Vis Sci. 2006; 47:909-916.
46. Utheim T P, Raeder S, Utheim O A, et al. A novel method for preserving cultured limbal epithelial cells. Br J Ophthalmol. 2007; 91:797-800.
47. James S E, Rowe A, Ilari L, Daya S, Martin R. The potential for eye bank limbal rings to generate cultured corneal epithelial allografts. Cornea. 2001; 20:488-494.
48. Joseph A, Powell-Richards A O, Shanmuganathan V A, Dua H S. Epithelial cell characteristics of cultured human limbal explants. Br J Ophthalmol. 2004; 88:393-398.
49. Shanmuganathan V A, Rotchford A P, Tullo A B, Joseph A, Zambrano I, Dua H S. Epithelial proliferative potential of organ cultured corneoscleral rims; implications for allo-limbal transplantation and eye banking. Br J Ophthalmol. 2006; 90:55-58.
50. Zito-Abbad E, Borderie V M, Baudrimont M, et al. Corneal epithelial cultures generated from organ-cultured limbal tissue: factors influencing epithelial cell growth. Curr Eye Res. 2006; 31:391-399.
51. Summerlin W T, Miller G E, Harris J E, Good R A. The organ-cultured cornea: an in vitro study. Invest Ophthalmol. 1973; 12:176-180.
52. Lass J H, Gordon J F, Sugar A, et al. Optisol containing streptomycin. Am J. Ophthalmol. 1993; 116:503-504.
53. Tungsiripat T, Sarayba M A, Taban M, Sweet P M, Osann K E, Chuck R S. Viability of limbal epithelium after anterior lamellar harvesting using a microkeratome. Ophthalmology. 2004; I l 1:469-475.
54. Crewe J M, Armitage W J. Integrity of epithelium and endothelium in organ-cultured human corneas. Invest Ophthalmol Vis Sci. 2001; 42:1757-1761.
55. Komuro A, Hodge D O, Gores G J, Bourne W M. Cell death during corneal storage at 4 degrees C. Invest Ophthalmol Vis Sci. 1999; 40:2827-2832.
56. Chang S W, Wang Y H, Pang J H. The effects of epithelial viability on stromal keratocyte apoptosis in porcine comeas stored in Optisol-GS. Cornea. 2006; 25:78-84.
57. Lee S H, Tseng S C. Amniotic membrane transplantation for persistent epithelial defects with ulceration. Am J. Ophthalmol. 1997; 123:303-312.
58. Lauweryns B, van den Oord J J, Missotten L. The transitional zone between limbus and peripheral cornea. An immunohistochemical study. Invest Ophthalmol Vis Sci. 1993; 34:1991-1999.
59. Chen Z, de Paiva C S, Luo L, Kretzer F L, Pflugfelder S C, Li D Q. Characterization of putative stem cell phenotype in human limbal epithelia. Stem Cells. 2004; 22:355-366.
60. Livak K J, Schmittgen T D. Analysis of relative gene expression data using real-time quantitative PCR and the 2(-Delta Delta C(T)) Method. Methods. 2001; 25:402-408.
61. Duan W R, Garner D S, Williams S D, Funckes-Shippy C L, Spath I S, Blomme E A. Comparison of immunohistochemistry for activated caspase-3 and cleaved cytokeratin 18 with the TUNEL method for quantification of apoptosis in histological sections of PC-3 subcutaneous xenografts. J Pathol. 2003; 199:221-228.
62. Borderie V M, Kantelip B M, Delbosc B Y, Oppermann M T, Laroche L. Morphology, histology, and ultrastructure of human C31 organ-cultured corneas. Cornea. 1995; 14:300-310.
63. van der Want H J, Pels E, Schuchard Y, Olesen B, Sperling S. Electron microscopy of cultured human corneas. Osmotic hydration and the use of a dextran fraction (dextran T 500) in organ culture. Arch Ophthalmol. 1983; 101:1920-1926.
64. Van Horn D L, Doughman D J, Harris J E, Miller G E, Lindstrom R, Good R A. Ultrastructure of human organ-cultured cornea. II. Stroma and epithelium. Arch Ophthalmol. 1975; 93:275-277.
65. Lindstrom R L, Doughman D J, Van Horn D L, Dancil D, Harris J E. A metabolic and electron microscopic study of human organ-cultured cornea. Am J. Ophthalmol. 1976; 82:72-82.
66. Means T L, Geroski D H, L'Hernault N, Grossniklaus H E, Kim T, Edelhauser H F. The corneal epithelium after optisol-GS storage. Cornea. 1996; 15:599-605.
67. Moore J E, McMullen C B, Mahon G, Adamis A P. The corneal epithelial stem cell. DNA Cell Biol. 2002; 21:443-451.
68. Hernandez Galindo E E, Theiss C, Steuhl K P, Meller D. Expression of Delta Np63 in response to phorbol ester in human limbal epithelial cells expanded on intact human amniotic membrane. Invest Ophthalmol Vis Sci. 2003; 44:2959-2965.
69. Hsuch Y J, Wang D Y, Cheng C C, Chen J K. Age-related expressions of p63 and other keratinocyte stem cell markers in rat cornea. J Biomed Sci. 2004; 11:641-651.
70. Kasper M, Moll R, Stosiek P, Karsten U. Patterns of cytokeratin and vimentin expression in the human eye. Histochemistry. 1988; 89:369-377.
71. Lauweryns B, van den Oord J J, De V R, Missotten L. A new epithelial cell type in the human cornea. Invest Ophthalmol Vis Sci. 1993; 34:1983-1990.
72. Schermer A, Galvin S, Sun T T. Differentiation-related expression of a major 64K corneal keratin in vivo and in culture suggests limbal location of corneal epithelial stem cells. J. Cell Biol. 1986; 103:49-62.
73. Chen Z, Evans W H, Pflugfelder S C, Li D Q. Gap junction protein connexin 43 serves as a negative marker for a stem cell-containing population of human limbal epithelial cells. Stem Cells. 2006; 24:1265-1273.
74. Matic M, Petrov 1N, Chen S, Wang C, Dimitrijevich S D, Wolosin J M. Stem cells of the corneal epithelium lack connexins and metabolite transfer capacity. Differentiation. 1997; 61:251-260.
75. Wolosin J M, Xiong X, Schutte M, Stegman Z, Tieng A. Stem cells and differentiation stages in the limbo-corneal epithelium. Prog Retin Eye Res. 2000; 19:223-255.
76. Kurpakus M A, Maniaci M T, Esco M. Expression of keratins K12, K4 and K14 during development of ocular surface epithelium. Curr Eye Res. 1994; 13:805-814.
77. Barnard Z, Apel A J, Harkin D G. Phenotypic analyses of limbal epithelial cell cultures derived from donor corneoscleral rims. Clin Experiment Ophthalmol. 2001; 29:138-142.
78. Scott R A, Lauweryns B, Snead D M, Haynes R J, Mahida Y, Dua H S. E-cadherin distribution and epithelial basement membrane characteristics of the normal human conjunctiva and cornea. Eye. 1997; 11:607-612.
79. Kerr J F, Wyllie A H, Currie A R. Apoptosis: a basic biological phenomenon with wide-ranging implications in tissue kinetics. Br J. Cancer. 1972; 26:239-257.
80. Metzstein M M, Stanfield G M, Horvitz H R. Genetics of programmed cell death in C. elegans: past, present and future. Trends Genet. 1998; 14:410-416.
81. Stehlik C, Hayashi H, Pio F, Godzik A, Reed J C. CARD6 is a modulator of NF-kappa B activation by Nodl- and Cardiak-mediated pathways. J Biol. Chem. 2003; 278:31941-31949.
82. Baeuerle P A, Baltimore D. NF-kappa B: ten years after. Cell. 1996; 87:13-20.
83. Barnes P J, Karin M. Nuclear factor-kappaB: a pivotal transcription factor in chronic inflammatory diseases. N Engl J of Med. 1997; 336:1066-1071.
84. Kopp E B, Ghosh S, NF-kappa B and rel proteins in innate immunity. Adv Immunol. 1995; 58:1-27.
85. Van Antwerp D J, Martin S J, Kafri T, Green D R, Verma I M. Suppression of TNF-alpha-induced apoptosis by NF-kappaB. Science. 1996; 274:787-789.
86. Wang C Y, Mayo M W, Baldwin A S, Jr. TNF- and cancer therapy-induced apoptosis: potentiation by inhibition of NF-kappaB. Science. 1996; 274:784-787.
87. Beg A A, Baltimore D. An essential role for NF-kappaB in preventing TNF-alpha-induced cell death. Science. 1996; 274:782-784.
88. Salvesen G S, Dixit V M. Caspases: intracellular signaling by proteolysis. Cell. 1997; 91:443-446.
89. Yuan J. Transducing signals of life and death. Curr Opin Cell Biol. 1997; 9:247-251.
90. Wallach D, Varfolomeev E E, Malinin N L, Goltsev Y V, Kovalenko A V, Boldin M P. Tumor necrosis factor receptor and Fas signaling mechanisms. Annu Rev Immunol. 1999; 17:331-367.
91. Reed J C. Cytochrome c: can't live with it—can't live without it. Cell. 1997; 91:559-562.
92. Green D R, Reed J C. Mitochondria and apoptosis. Science. 1998; 281:1309-1312.
93. Reed J C. Double identity for proteins of the Bcl-2 family. Nature. 1997; 387:773-776.
94. Yamamoto K, Ladage P M, Ren D H, et al. Bcl-2 expression in the human cornea. Exp Eye Res. 2001; 73:247-255.
95. Richard N R, Anderson J A, Weiss J L, Binder P S. Air/liquid corneal organ culture: a light microscopic study. Curr Eye Res. 1991; 10:739-749.
96. Raeder S, Utheim T P, Utheim O A et al. Effects of Organ Culture and Optisol-GS Storage on Structural Integrity, Phenotypes, and Apoptosis in Cultured Corneal Epithelium. Invest Ophthalmol. Vis. Sci. 2007; 48:5484-93.
97. Raeder S, Utheim T P, Utheim O A et al. Effect of limbal explant orientation on the histology, phenotype, ultrastructure and barrier function of cultured limbal epithelial cells. Acta Ophthalmol. Scand. 2007; 85:377-86.
98. Schlotzer-Schrehardt U, Kruse F E. Identification and characterization of limbal stem cells. [Review][128 refs]. 2005; 81:247-64.
99. Figueira E C, Di G N, Coroneo M T et al. The phenotype of limbal epithelial stem cells. 2007.
100. Gain P, Thuret G, Chiquet C et al. Use of a pair of blood culture bottles for sterility testing of corneal organ culture media. British Journal of Ophthalmology 2001; 85:1158-62.
101. Thuret G, Carricajo A, Chiquet C et al. Sensitivity and rapidity of blood culture bottles in the detection of cornea organ culture media contamination by bacteria and fungi. Br. J. Ophthalmol. 2002; 86:1422-7.
102. Utheim T P, Raeder S, Utheim O A et al. A novel method for preserving cultured limbal epithelial cells. Br. J. Ophthalmol. 2006.
103. Wiley, L., SundarRaj, N., Sun, T. T. & Thoft, R. A. Regional heterogeneity in human corneal and limbal epithelia: an immunohistochemical evaluation. 32, 594-602 (1991).
104. Haskjold, E., Refsum, S. B. & Bjerknes, R. Cell renewal of the rat corneal epithelium. A method to compare corresponding corneal areas from individual animals. (1988).
105. Haskjold, E., Bjerknes, R. & Refsum, S. B. Cell kinetics during healing of corneal epithelial wounds. (1989).
106. Short, K. M. & Cox, T. C. Subclassification of the RBCC/TRIM superfamily reveals a novel motif necessary for microtubule binding. (2006).
107. Kawai, S., Yamauchi, M., Wakisaka, S., Ooshima, T. & Amano, A.
Zinc-finger transcription factor odd-skipped related 2 is one of the regulators in osteoblast proliferation and bone formation. (2007).
108. Lan, Y. et al. Odd-skipped related 2 (Osr2) encodes a key intrinsic regulator of secondary palate growth and morphogenesis. (2004).
109. Lan, Y., Wang, Q., Ovitt, C. E. & Jiang, R. A unique mouse strain expressing Cre recombinase for tissue-specific analysis of gene function in palate and kidney development. (2007).
110. Notarnicola, C., Le, G. L., Fort, P., Faure, S. & de Santa, B. P. Dynamic expression patterns of RhoV/Chp and RhoU/Wrch during chicken embryonic development. (2008).
111. Ory, S., Brazier, H. & Blangy, A. Identification of a bipartite focal adhesion localization signal in RhoU/Wrch-1, a Rho family GTPase that regulates cell adhesion and migration. (2007).
112. Buschke, W., Friedenwald, J. & Fleischmann, W. Studies on the miotic activity of the corneal epithelium. Methods. The effetcts of Colchicine, Ether, Cocaine and Ephedrin. Bulletin of the Johns Hopkins Hospital, 143-169. 1943.
113. Kaufman B, Gay H & Hollaender A. Distribution of mitoses in the corneal epithelium. Anat. Rec. 90, 161-178. 1944.

1-91. (canceled)

92. A method of storing cells or tissue comprising keeping the cells or tissue at a temperature of between 3° C. and 37° C. for a period of at least two days during which there is substantially no increase in the differentiation of the cells wherein the cells or tissue are selected from the group consisting of: limbal epithelial cells, conjunctival cells, corneal endothelial cells, retinal cells, mucosal cells, epidermal cells and bone marrow derived cells.

93. A method according to claim 92 wherein the limbal epithelial cells, conjunctival cells, corneal endothelial cells, retinal cells, mucosal cells, epidermal cells or bone marrow derived cells are cultured cells.

94. A method according to claim 92, comprising keeping, the cells or tissue at a temperature of between 3° C. and 30° C.

95. A method according to claim 94 comprising keeping the cells or tissue at a temperature between 18° C. and 28° C.

96. A method according to claim 95 comprising keeping the cells or tissue at a temperature between 20° C. and 25° C.

97. A method according to claim 96 comprising keeping the cells or tissue at a temperature between 22° C. and 24° C.

98. A method according to claim 97 comprising keeping the cells or tissue at a temperature between 22° C. and 23° C.

99. A method according to claim 92 comprising keeping the cells or tissue at the temperature for a period of at least three days.

100. A method according to claim 99 comprising keeping the cells or tissue at the temperature for at least four days.

101. A method according to claim 100 comprising keeping the cells or tissue at the temperature for at least seven days.

102. A method according to claim 92 wherein the cells or tissue are submerged within a liquid medium.

103. A method according to claim 102 wherein the liquid medium comprises minimal essential medium.

104. A method according to 102 wherein the liquid medium is a serum-free medium.

105. A method according to claim 104 wherein the medium comprises a constituent selected from Optisol-GS and PAA-Quantum.

106. A method according to claim 104 wherein the serum-free medium comprises: a buffering agent and minimal essential medium.

107. A method according to claim 106 wherein the buffering agent comprises HEPES (4-(2-hydroxyethyl)-1-piperazineethane-sulfonic acid).

108. A method according to claim 107 wherein the HEPES is at 25 mM concentration.

109. A method according to claim 106 wherein the minimal essential medium comprises amino acids, salts, glucose and vitamins.

110. A method according to claim 109, wherein the salts comprise at least one of potassium chloride, magnesium sulphate, sodium chloride and sodium dihydrogen phosphate.

111. A method according to claim 109 wherein the vitamins comprise at least one of folic acid, nicotinamide, riboflavin and B-12.

112. A method according to claim 102 wherein the liquid medium comprises sodium bicarbonate.

113. A method according to claim 92 further comprising the step of culturing the cells or tissue, prior to storage.

114. A kit for storing cells or tissue comprising:
a frame having an opening therein and a peripheral wall surrounding the opening;
a sealable receptacle for receiving the frame and for receiving a liquid medium, a section of the sealable receptacle being formed of a resilient member for permitting access to the interior of the receptacle by a penetrating element and subsequently forming a seal after withdrawal of the penetrating element; and
at least one float attachable to the frame for supporting the frame in the medium.

115. A kit according to claim 114 further comprising an elongate or annular resilient element locatable about the peripheral wall for securing a substrate across the opening in the frame.

116. A kit according to claim 114 wherein the float is locatable on the frame such that a substrate supported on the frame will lie less than 2 mm below the level of the liquid medium.

117. A kit according to claim 116 wherein the float is locatable on the frame such that a substrate supported on the frame will lie less than 1 mm below the level of the liquid medium.

118. A kit according to claim 114 wherein the peripheral wall comprises one or more apertures for allowing passage of the medium therethrough.

119. A kit according to claim 114 further comprising the medium.

120. A kit according to claim 119 wherein the medium is a serum-free medium.

121. A kit according to claim 120 wherein the constituent for the medium is selected from Optisol-GS and PAA-Quantum.

122. A method of using a kit according to claim 114 for storing cells or tissue.

123. A method according to claim 92 using the kit of claim 114.
